(19) United States
(12) Patent Application Publication
(10) Pub. No.: US 2016/0287061 A1
(43) Pub. Date: Oct. 6, 2016

(54) ENDOSCOPE SYSTEM, PROCESSOR DEVICE, AND OPERATION METHOD OF ENDOSCOPE SYSTEM

(71) Applicant: FUJIFILM Corporation, Tokyo (JP)

(72) Inventor: Norimasa SHIGETA, Ashigarakami-gun (JP)

(73) Assignee: FUJIFILM Corporation, Tokyo (JP)

(21) Appl. No.: 15/044,914

(22) Filed: Feb. 16, 2016

(30) Foreign Application Priority Data
Mar. 31, 2015 (JP) 2015-073463

(51) Int. Cl. A61B 1/06; G06T 1/20; H04N 5/225; A61B 5/1455; A61B 1/00

(52) U.S. Cl. CPC A61B 1/0669; A61B 5/14551; A61B 1/0638; A61B 1/0002; A61B 1/00018; A61B 1/00009; H04N 5/225; G06T 1/20; H04N 2005/2255

(57) ABSTRACT

There are provided an endoscope system, a processor device, and an operation method of an endoscope system that can calculate an accurate oxygen saturation by reducing artifacts. An endoscope system includes: an oxygen saturation correction unit that calculates the amount of change in oxygen saturation in the latest second period with respect to the oxygen saturation in the past first period and that maintains a value of the oxygen saturation in the second period in a case in which the amount of change is less than a threshold value and corrects the oxygen saturation in the second period to a value obtained as a result of setting the amount of change to the threshold value in a case in which the amount of change is equal to or greater than the threshold value; and an oxygen saturation image generation unit that generates an oxygen saturation image using the oxygen saturation in the second period.

### CROSS-REFERENCE TO RELATED APPLICATIONS

The present application claims priority under 35 U.S.C. §119 to Japanese Patent Application No. 2015-073463, filed on Mar. 31, 2015. The above application(s) is hereby expressly incorporated by reference, in its entirety, into the present application.

### BACKGROUND OF THE INVENTION

1. Field of the Invention

The present invention relates to an endoscope system, a processor device, and an operation method of an endoscope system for calculating the oxygen saturation of an observation target.

2. Description of the Related Art

In the medical field, diagnosis using an endoscope system including a light source device, an endoscope, and a processor device has been widely performed. The endoscope includes an insertion unit that is inserted into a subject, and images an observation target (for example, a mucosa in the subject) irradiated with illumination light generated by the light source device. The processor device generates an image of the observation target using an image signal obtained by imaging the observation target, and displays the image on a monitor.

In addition, an endoscope system that acquires not only an image of the observation target but also information indicating the characteristics of the observation target has been known in recent years. For example, in the endoscope system disclosed in JP2013-022341A, the oxygen saturation is measured as a characteristic of the observation target by irradiating the observation target with oxygen saturation measurement light (hereinafter, referred to as measurement light) having a wavelength band in which there is a difference between the absorption coefficient of oxygenated hemoglobin and the absorption coefficient of reduced hemoglobin, and an image showing the oxygen saturation (hereinafter, referred to as an oxygen saturation image) is generated and displayed.

### SUMMARY OF THE INVENTION

The oxygen saturation image is generated by superimposing the information of the oxygen saturation on an image generated from the image signal showing the structural characteristics, such as the shape of the observation target. Therefore, in order to generate an oxygen saturation image, an image signal for showing the shape or the like of the observation target by emitting white light or the like and an image signal for calculating the oxygen saturation by emitting the measurement light are required. For this reason, imaging of two frames is typically required.

On the other hand, in the observation target of the endoscope system, there is a movement due to peristalsis or the like. Accordingly, even if there is no movement in the observation target, there is a relative movement with respect to the endoscope in the observation target due to the movement of the endoscope. It is not possible to stop such movement of the observation target. Accordingly, when the imaging of two frames is performed as described above in order to generate and display an oxygen saturation image, a pseudo change (hereinafter, referred to as artifacts) in the oxygen saturation value due to the random noise difference between image signals of two frames or the movement of the observation target (or the endoscope) may occur.

The oxygen saturation changes depending on the characteristics (medical conditions or the like) of the observation target, but the acquisition interval of an image signal is milliseconds. Accordingly, the oxygen saturation hardly changes in a short time to obtain an image signal of two frames. However, artifacts of the oxygen saturation change with the movement of the observation target or the like as described above. Therefore, if there are artifacts, there may be a portion for which an inaccurate oxygen saturation that does not fit the actual conditions of a large change is calculated.

It is an object of the invention to provide an endoscope system, a processor device, and an operation method of an endoscope system that can calculate an accurate oxygen saturation by reducing artifacts, which are an error of the value of the oxygen saturation corresponding to an instantaneous abrupt change of the observation target while leaving the characteristic change of the observation target or the original temporal or spatial change (for example, the movement of a position in the image) in the oxygen saturation corresponding to the long-term movement of the observation target, which is longer than the interval of imaging.

An endoscope system of the invention includes: an image signal acquisition unit that acquires a first period image signal by imaging an observation target, which is irradiated with illumination light, in a first period and that acquires a second period image signal by imaging the observation target in a second period after acquiring the first period image signal; an oxygen saturation calculation unit that calculates an oxygen saturation of the observation target in the first period using the first period image signal and that calculates an oxygen saturation of the observation target in the second period using the second period image signal; an oxygen saturation correction unit that calculates an amount of change in an oxygen saturation in the second period with respect to an oxygen saturation in the first period and that maintains a value of the oxygen saturation in the second period in a case in which the amount of change is less than a threshold value and corrects the oxygen saturation in the second period to a value obtained as a result of setting the amount of change to the threshold value in a case in which the amount of change is equal to or greater than the threshold value; and an oxygen saturation image generation unit that generates an oxygen saturation image showing an oxygen saturation of the observation target using the oxygen saturation in the second period.

It is preferable that the oxygen saturation correction unit sets the threshold value based on a distribution of the oxygen saturation in the first period.

It is preferable to further include: a multi-resolution processing unit that generates a plurality of multi-resolution image signals having different resolutions by performing multi-resolution processing on the second period image signal; and a combination processing unit that generates a second period composite image signal by applying a weighting to each of the plurality of multi-resolution image signals. Preferably, the oxygen saturation calculation unit calculates the oxygen saturation in the second period using the second period composite image signal.

Preferably, the second period image signal includes a first group image signal acquired at a first timing of the second period and a second group image signal acquired at a second timing, which is different from the first timing, of the second period, and the combination processing unit compares a plurality of multi-resolution image signals generated from the first group image signal with a plurality of multi-resolution image signals generated from the second group image signal for each resolution, and performs combination processing by setting a larger weighting as the multi-resolution image signals having resolutions in which a difference between the multi-resolution image signal generated from the first group image signal and the multi-resolution image signal generated from the second group image signal is smaller.

Preferably, each of the first and second group image signals includes a green image signal corresponding to a green wavelength band and a red image signal corresponding to a red wavelength band, and the combination processing unit compares ratios between the green image signal and the red image signal after multi-resolution processing for each resolution and sets the weighting.

It is preferable to further include a multi-resolution processing unit that generates a plurality of multi-resolution image signals having different resolutions by performing multi-resolution processing on the second period image signal. Preferably, the oxygen saturation calculation unit calculates the oxygen saturation in the second period using the multi-resolution image signal having a specific resolution of the plurality of multi-resolution image signals.

Preferably, the second period image signal includes a first group image signal acquired at a first timing of the second period and a second group image signal acquired at a second timing, which is different from the first timing, of the second period, and the oxygen saturation calculation unit calculates the oxygen saturation in the second period using the multi-resolution image signals in which a difference between the multi-resolution image signal generated from the first group image signal and the multi-resolution image signal generated from the second group image signal is the smallest and resolutions are the highest.

It is preferable to further include: an oxygen saturation storage unit that stores a plurality of oxygen saturations including the oxygen saturation in the first period and calculated before the second period; and a noise reduction unit that reduces noise of the oxygen saturation in the second period using a plurality of oxygen saturations stored in the oxygen saturation storage unit.

It is preferable to further include a movement detection unit that detects a relative movement between the observation target and an endoscope. Preferably, the noise reduction unit increases the number of oxygen saturations used for reduction of the noise as the movement detected by the movement detection unit increases.

It is preferable to further include a position shift correction unit that corrects a position shift between the oxygen saturation in the second period and the oxygen saturation in the first period.

A processor device of the invention includes: an image signal acquisition unit that acquires a first period image signal by imaging an observation target, which is irradiated with illumination light, in a first period and that acquires a second period image signal by imaging the observation target in a second period after acquiring the first period image signal; an oxygen saturation calculation unit that calculates an oxygen saturation of the observation target in the first period using the first period image signal and that calculates an oxygen saturation of the observation target in the second period using the second period image signal; an oxygen saturation correction unit that calculates an amount of change in an oxygen saturation in the second period with respect to an oxygen saturation in the first period and that maintains a value of the oxygen saturation in the second period in a case in which the amount of change is less than a threshold value and corrects the oxygen saturation in the second period to a value obtained as a result of setting the amount of change to the threshold value in a case in which the amount of change is equal to or greater than the threshold value; and an oxygen saturation image generation unit that generates an oxygen saturation image showing an oxygen saturation of the observation target using the oxygen saturation in the second period.

An operation method of an endoscope system of the invention includes: a step of causing an image signal acquisition unit to acquire a first period image signal by imaging an observation target, which is irradiated with illumination light, in a first period and to acquire a second period image signal by imaging the observation target in a second period after acquiring the first period image signal; a step of causing an oxygen saturation calculation unit to calculate an oxygen saturation of the observation target in the first period using the first period image signal and to calculate an oxygen saturation of the observation target in the second period using the second period image signal; a step of causing an oxygen saturation correction unit to calculate an amount of change in an oxygen saturation in the second period with respect to an oxygen saturation in the first period and to maintain a value of the oxygen saturation in the second period in a case in which the amount of change is less than a threshold value and correct the oxygen saturation in the second period to a value obtained as a result of setting the amount of change to the threshold value in a case in which the amount of change is equal to or greater than the threshold value; and a step of causing an oxygen saturation image generation unit to generate an oxygen saturation image showing an oxygen saturation of the observation target using the oxygen saturation in the second period.

In the endoscope system, the processor device, and the operation method of an endoscope system of the invention, in a case in which the amount of change in oxygen saturation is less than the threshold value and the change in oxygen saturation can be regarded as a temporal or spatial change in original oxygen saturation corresponding to the characteristic change of the observation target, the value of the calculated oxygen saturation is maintained. In a case in which the amount of change in oxygen saturation is equal to or greater than the threshold value and a possibility that the change in oxygen saturation will be artifacts is high, the value of the oxygen saturation is corrected to a value obtained as a result of setting the amount of change to the threshold value. In this manner, the change in oxygen saturation due to artifacts is reduced. Therefore, in the endoscope system, the processor device, and the operation method of an endoscope system of the invention, it is possible to calculate an accurate oxygen saturation by reducing artifacts while leaving a temporal or spatial change in original oxygen saturation corresponding to the characteristic change of the observation target.

### BRIEF DESCRIPTION OF THE DRAWINGS

FIG. 1 is an external view of an endoscope system.

FIG. 2 is a block diagram showing a function of the endoscope system.

FIG. 3 is a graph showing the spectra of violet light, blue light, green light, and red light.

FIG. 4 is an explanatory diagram showing the configuration of a band limiting unit.

FIG. 5 is a graph showing the spectrum of blue light for normal observation.

FIG. 6 is a graph showing the spectrum of illumination light in a normal observation mode.

FIG. 7 is a graph showing the spectrum of measurement light.

FIG. 8 is a graph showing the absorption coefficients of oxygenated hemoglobin and reduced hemoglobin.

FIG. 9 is a graph showing the spectrum of illumination light in a second light emission mode.

FIG. 10 is a graph showing the spectral characteristics of a color filter.

FIG. 11 is a timing chart showing the acquisition timing of an image signal in an oxygen saturation observation mode.

FIG. 12 is a block diagram of an oxygen saturation image generation section.

FIG. 13 is a graph showing a correlation between the signal ratio and the oxygen saturation.

FIG. 14 is an explanatory diagram showing a function of an oxygen saturation correction section.

FIG. 15 is an explanatory diagram showing a method of correcting the oxygen saturation.

FIG. 16 is an explanatory diagram showing a method of correcting the oxygen saturation.

FIG. 17 shows a base image.

FIG. 18 shows the oxygen saturation in a second period that is obtained as a result of correcting a change equal to or greater than a threshold value to the threshold value.

FIG. 19 shows an oxygen saturation image.

FIG. 20 shows a flowchart of a first embodiment.

FIG. 21 shows an oxygen saturation image in a case in which the oxygen saturation is not corrected.

FIG. 22 shows a G1 image signal with a dark region.

FIG. 23 shows an oxygen saturation image that is generated using an image signal with a dark region.

FIG. 24 is a block diagram of a special processing section of a second embodiment.

FIG. 25 is an explanatory diagram of multi-resolution processing.

FIG. 26 is an explanatory diagram of combination processing.

FIG. 27 is an explanatory diagram showing a process of setting the weighting in combination processing.

FIG. 28 is a block diagram of a special processing section in a modification example.

FIG. 29 is a block diagram of a special processing section in a modification example.

FIG. 30 is a schematic diagram of a capsule endoscope.

### DESCRIPTION OF THE PREFERRED EMBODIMENTS

### First Embodiment

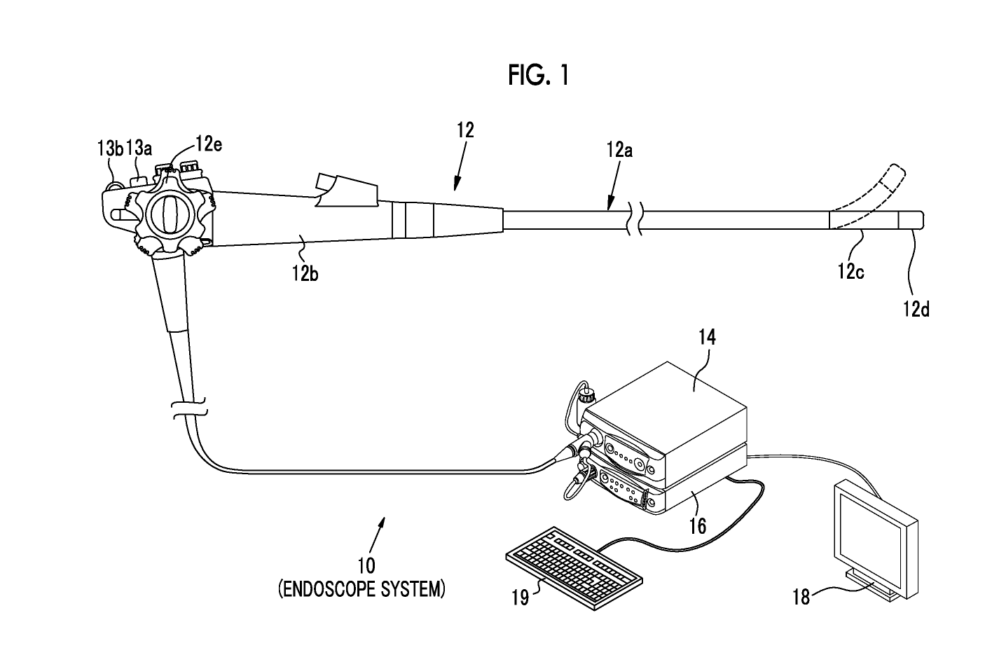

As shown in FIG. 1, an endoscope system 10 includes an endoscope 12, a light source device 14, a processor device 16, a monitor 18, and a console 19. The endoscope 12 is optically connected to the light source device 14, and is electrically connected to the processor device 16. The endoscope 12 includes an insertion unit 12a that is inserted into a subject, an operation unit 12b provided at the proximal end of the insertion unit 12a, and a bending portion 12c and a distal portion 12d that are provided at the distal side of the insertion unit 12a. By operating an angle knob 12e of the operation unit 12b, the bending portion 12c is bent. Through the bending operation, the distal portion 12d is directed toward a desired direction.

In addition to the angle knob 12e, a mode selector switch 13a and a zoom operation unit 13b are provided in the operation unit 12b. The mode selector switch 13a is used for an observation mode switching operation. The endoscope system 10 has a normal observation mode and an oxygen saturation observation mode as observation modes. In the normal observation mode, an image of natural colors obtained by imaging an observation target with white light (hereinafter, referred to as a normal image) is displayed on the monitor 18. In the oxygen saturation observation mode, the oxygen saturation of the observation target is measured by irradiating the observation target with measurement light having a specific wavelength band for measuring the oxygen saturation, and an oxygen saturation image that is colored using the value of the oxygen saturation is displayed on the monitor 18.

The processor device 16 is electrically connected to the monitor 18 and the console 19. The monitor 18 outputs and displays an image, image information to be attached to the image, or the like in each observation mode. The console 19 functions as a user interface for receiving an input operation, such as a function setting. In addition, an external recording unit (not shown) in which an image, image information, or the like is recorded may be connected to the processor device 16.

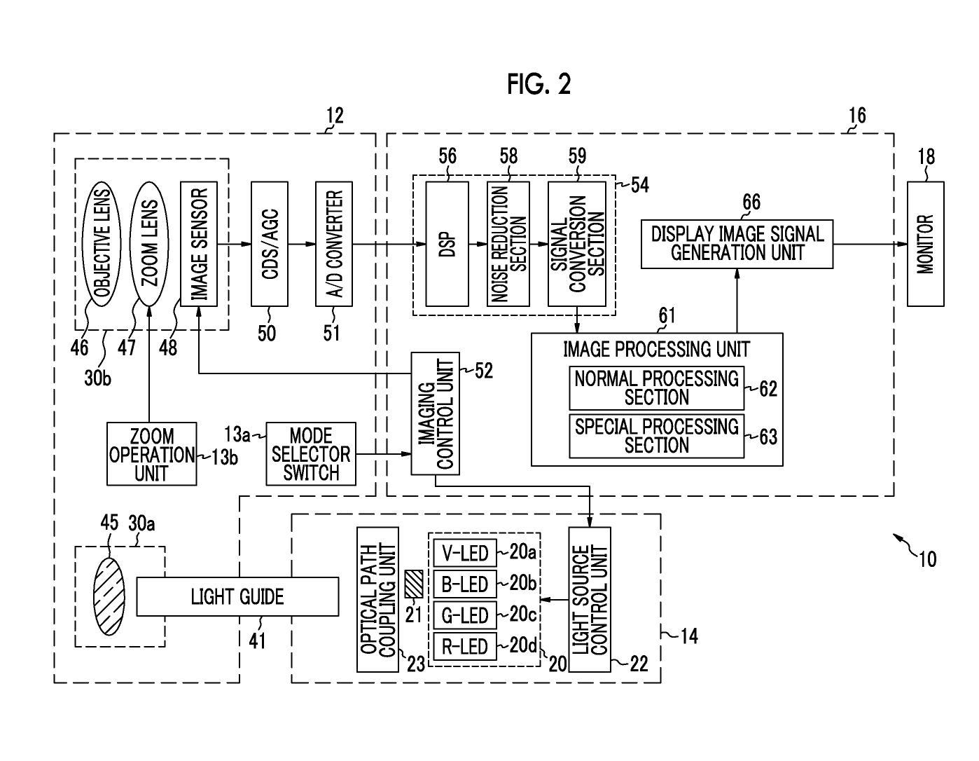

As shown in FIG. 2, the light source device 14 includes four semiconductor light sources, and includes a light source unit 20 that generates light emitted to the observation target, a band limiting unit 21 that limits the wavelength band of the light emitted from the light source unit 20, a light source control unit 22 that controls the driving of the light source unit 20 and the band limiting unit 21, and an optical path coupling unit 23 that couples the optical paths of light components generated by the light source unit 20 and the band limiting unit 21.

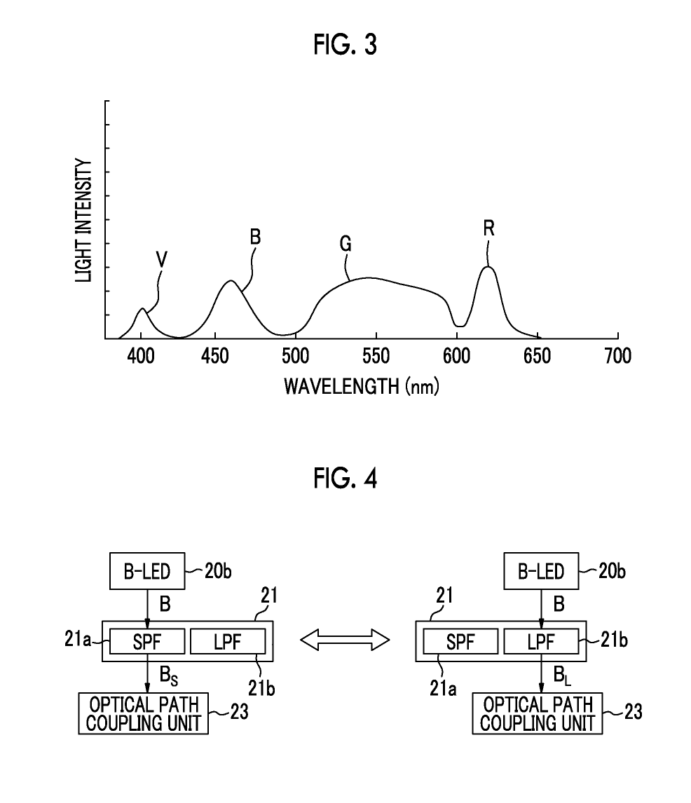

The light source unit 20 includes LEDs of four colors of a violet light emitting diode (V-LED) 20a, a blue light emitting diode (B-LED) 20b, a green light emitting diode (G-LED) 20c, and a red light emitting diode (R-LED) 20d. As shown in FIG. 3, the V-LED 20a is a violet light source that emits violet light V in a wavelength band of 380 nm to 420 nm that has a center wavelength of 405 nm. The B-LED 20b is a blue light source that emits blue light B in a wavelength band of 420 nm to 500 nm that has a center wavelength of 460 nm. The G-LED 20c is a green light source that emits green light G in a wavelength band of 480 nm to 600 nm. The R-LED 20d is a red light source that emits red light R in a wavelength band of 600 nm to 650 nm that has a center wavelength of 620 nm to 630 nm. In addition, the width of the center wavelength of each of the V-LED 20a and the B-LED 20b is about ±5 nm to ±10 nm.

The band limiting unit 21 is provided on the optical path of the B-LED 20b, and generates light having a specific wavelength band from the blue light emitted from the B-LED 20b. Specifically, as shown in FIG. 4, the band limiting unit 21 includes a short pass filter (SPF) 21a and a long pass filter (hereinafter, referred to as an LPF) 21b that are freely switched. Switching between the SPF 21a and LPF 21b is controlled by the light source control unit 22.

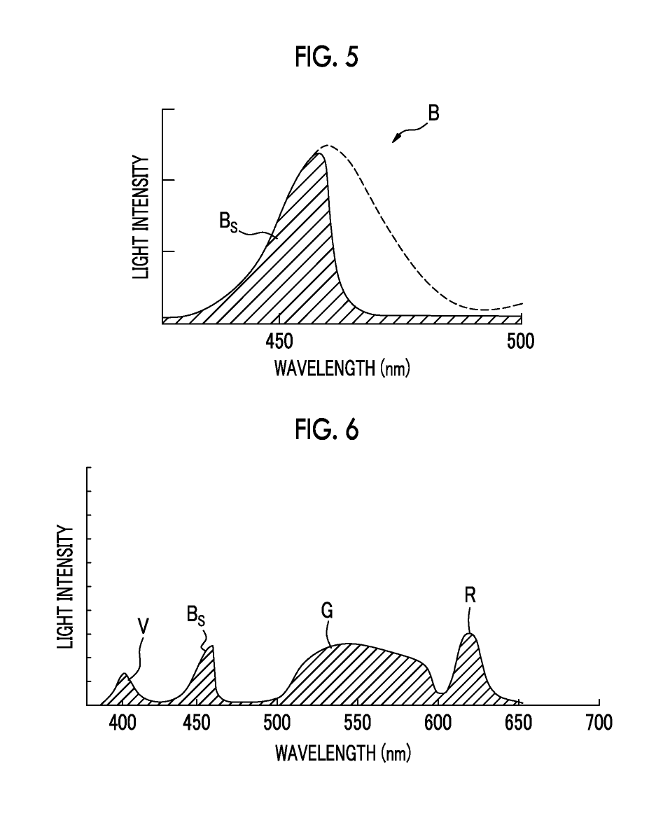

As shown in FIG. 5, the SPF 21a transmits the short wavelength side wavelength band (less than the wavelength of 460 nm) of the blue light B emitted from the B-LED 20b, and cuts the long wavelength side wavelength band (equal to or greater than the wavelength of 460 nm) of the blue light B emitted from the B-LED 20b. Accordingly, the SPF 21a generates blue light for normal observation B_{S} from the blue light B. For example, in the case of the normal observation mode, the light source control unit 22 places the SPF 21a in the optical path of the B-LED 20b, and turns on all of the V-LED 20a, the B-LED 20b, the G-LED 20c, and the R-LED 20d. Therefore, as shown in FIG. 6, the violet light V, the blue light for normal observation B_{S}, the green light and the red light R are coupled by the optical path coupling unit 23, and the coupled light is emitted to the observation target as illumination light. The illumination light configured to include the violet light V, the blue light for normal observation B_{S}, the green light and the red light R is almost white light (hereinafter, referred to as white light for normal observation). The reason why the SPF 21a generates the blue light for normal observation B_{S} from the blue light B in the normal observation mode as described above is that the light in a wavelength band of about 460 nm to 500 nm reduces the contrast of structures, such as superficial blood vessels or a pit pattern.

In the present embodiment, in the normal observation mode, the V-LED 20a is turned on to irradiate the observation target with the white light for normal observation including the violet light V. However, the V-LED 20a may be turned off in the normal observation mode. In addition, the SPF 21a cuts the blue light B schematically at a wavelength of 460 nm. However, the actual cutting characteristic is a wavelength width of about 5 nm to 10 nm. For this reason, in order to cut the wavelength of 460 nm or more, the SPF 21a has a characteristic in which the transmittance is attenuated from the vicinity of the wavelength of 450 nm. In order to maintain the color rendering properties for a xenon light source, it is preferable that there is no discrete wavelength band in the spectrum of illumination light emitted to the observation target. Therefore, the SPF 21a has a cutting characteristic of reducing the amount of light in a wavelength band of 460 nm or more to the extent that the color rendering properties for the xenon light source can be maintained rather than reducing the amount of light in the wavelength band of 460 nm or more of the blue light B strictly to zero. For this reason, even if the SPF 21a is used, there is no discrete wavelength band in the illumination light emitted to the observation target (refer to FIGS. 5 and 6).

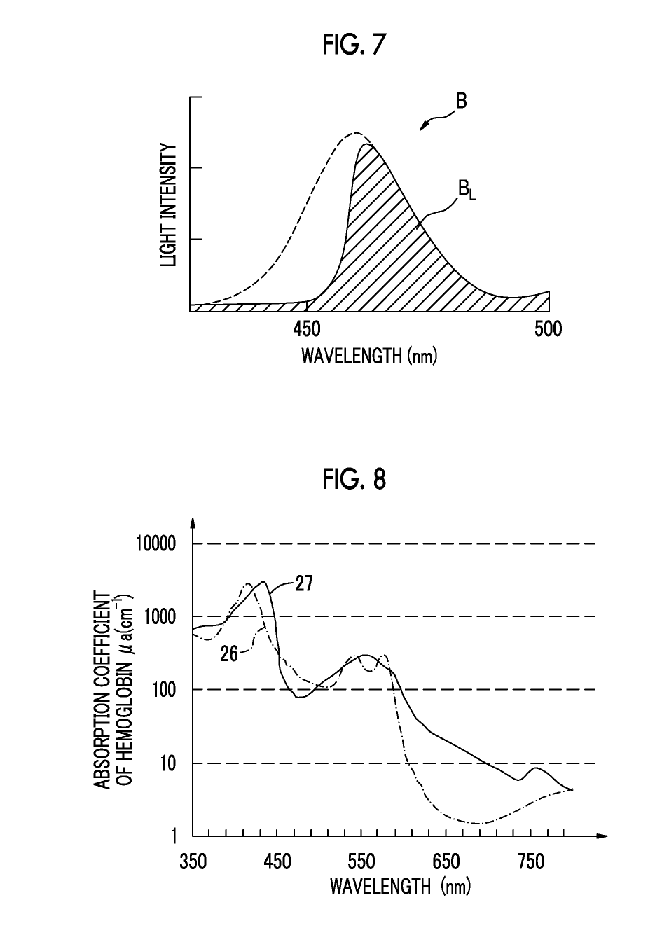

On the other hand, the LPF 21b is disposed on the optical path of the B-LED 20b in the oxygen saturation observation mode (refer to FIG. 4). As shown in FIG. 7, the LPF 21b cuts the short wavelength side wavelength band of the blue light B emitted from the B-LED 20b, and transmits the long wavelength side wavelength band of the blue light B emitted from the B-LED 20b. Accordingly, the LPF 21b generates measurement light B_{L} having a specific wavelength band for measuring the oxygen saturation from the blue light B.

The specific wavelength band for measuring the oxygen saturation is a wavelength band in which there is a difference between the absorption coefficient of oxygenated hemoglobin and the absorption coefficient of reduced hemoglobin to the extent that a difference in the amount of light absorption according to the oxygen saturation occurs. As shown in FIG. 8, the magnitude relationship between the absorption coefficient (graph 26) of oxygenated hemoglobin and the absorption coefficient (graph 27) of reduced hemoglobin differs depending on the wavelength band. The magnitude relationship may be reversed in multiple wavelength bands. For example, in the wavelength band from purple to blue, wavelengths at which the absorption coefficient of oxygenated hemoglobin and the absorption coefficient of reduced hemoglobin are the same are about 420 nm, about 450 nm, and about 500 nm. In the wavelength band of 420 nm to 450 nm, the absorption coefficient of oxygenated hemoglobin is smaller than the absorption coefficient of reduced hemoglobin. In the wavelength band of 450 nm to 500 nm, the absorption coefficient of oxygenated hemoglobin is larger than the absorption coefficient of reduced hemoglobin.

All of these wavelength bands can be used as specific wavelength bands for measuring the oxygen saturation. In the present embodiment, since the B-LED 20b emits the blue light B in the wavelength band of 420 nm to 500 nm, the LPF 21b transmits light having a wavelength of 460 nm or more to generate the measurement light B_{L}. Therefore, the measurement light B_{L} has a wavelength band of 460 nm to 500 nm in which the absorption coefficient of oxygenated hemoglobin is equal to or less than the absorption coefficient of reduced hemoglobin. If the SPF that transmits light having a wavelength band of 450 nm or less is used instead of the LPF 21b, the wavelength band of the measurement light B_{L} can be set to a wavelength band of 420 nm to 450 nm in which the absorption coefficient of oxygenated hemoglobin is equal to or greater than the absorption coefficient of reduced hemoglobin.

The blue light B emitted from the B-LED 20b includes a wavelength (isosbestic point) at which the absorption coefficient of oxygenated hemoglobin and the absorption coefficient reduced hemoglobin are the same. Therefore, the blue light B includes both of the wavelength band in which the absorption coefficient of oxygenated hemoglobin is equal to or greater than the absorption coefficient of reduced hemoglobin and the wavelength band in which the absorption coefficient of oxygenated hemoglobin is equal to or less than the absorption coefficient of reduced hemoglobin. For this reason, when the blue light B itself is used as measurement light for measuring the oxygen saturation, the measurement accuracy is low even though the oxygen saturation can be measured. Therefore, in the endoscope system 10, the measurement light B_{L} is generated by the LPF 21b of the band limiting unit 21.

In the present embodiment, the LPF 21b cuts a wavelength band less than the wavelength of 460 nm schematically. However, similar to the SPF 21a, the actual cutting characteristic is a wavelength width of about 5 nm to 10 nm. Accordingly, in order to cut reliably the short wavelength side wavelength band less than the wavelength of 450 nm, it is preferable that the LPF 21b has a characteristics in which the transmittance is attenuated from the vicinity of the wavelength of 460 nm as described above. This is because the isosbestic point, at which the absorption coefficient of oxygenated hemoglobin and the absorption coefficient of reduced hemoglobin are the same, is at the wavelength of 450 nm as described above. Thus, since the LPF 21b has a characteristic of cutting the short wavelength side wavelength band less than the wavelength of 450 nm, it is possible to calculate the oxygen saturation particularly accurately using the measurement light B_{L}.

In the oxygen saturation observation mode, the light source control unit 22 controls the light source unit 20 in first and second light emission modes. That is, the light source control unit 22 performs control for switching between the first and second light emission modes. In the first emission mode, the light source control unit 22 places the SPF 21a in the optical path of the B-LED 20b, and turns on all of the LEDs 20a to 20d of four colors. Therefore, in the first emission mode, in the same manner as in the normal observation mode, the normal white light for normal observation including the violet light V, blue light for observation Bs, the green light and the red light R is emitted to the observation target.

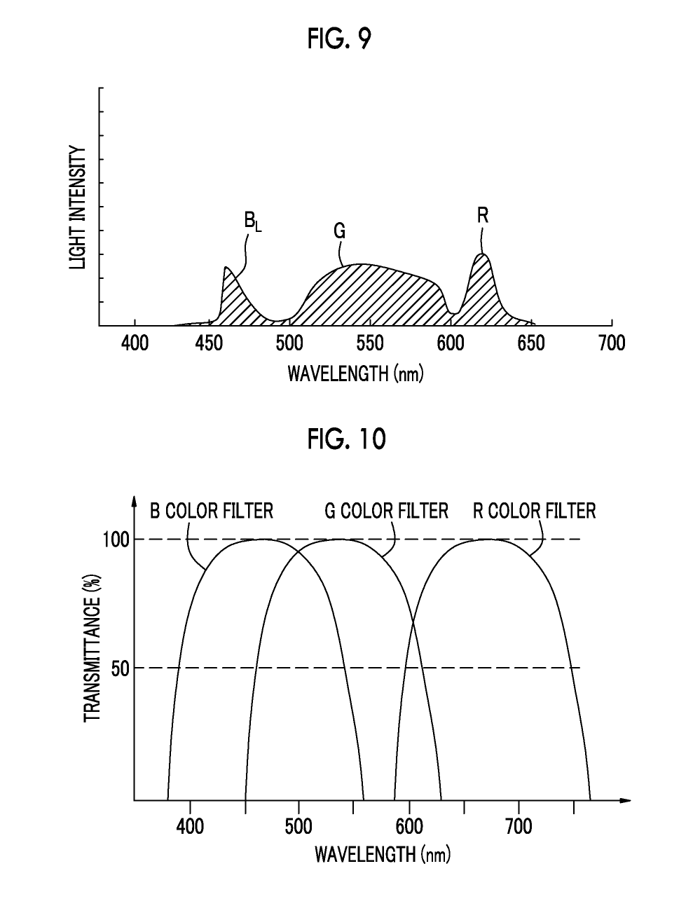

In the second emission mode, the light source control unit 22 places the LPF 21b in the optical path of the B-LED 20b, and turns off the V-LED 20a and turns on the B-LED 20b, the G-LED 20c, and the R-LED 20d. Therefore, as shown in FIG. 9, in the second emission mode, white light (hereinafter the white light for oxygen saturation measurement) configured to include the measurement light B_{L}, the green light and the red light R is emitted to the observation target.

The various illumination light components described above are incident on a light guide 41 through the optical path coupling unit 23. The light guide 41 is built into the endoscope 12 and a universal cord (cord for connecting the endoscope 12 to the light source device 14 and the processor device 16), and makes the illumination light guided from the optical path coupling unit 23 propagate to the distal portion 12d of the endoscope 12. As the light guide 41, it is possible to use a multi-mode fiber. As an example, it is possible to use a small-diameter fiber cable having a core diameter of 105 μm, a cladding diameter of 125 μm, and a diameter of φ0.3 mm to φ0.5 mm when a protective layer as an outer skin is included.

An illumination optical system 30a and an imaging optical system 30b are provided in the distal portion 12d of the endoscope 12. The illumination optical system 30a includes an illumination lens 45, and the illumination light propagated by the light guide 41 is emitted to the observation target through the illumination lens 45. The imaging optical system 30b includes an objective lens 46, a zoom lens 47, and an imaging sensor 48. The imaging sensor 48 images an observation target with light from the observation target through the objective lens 46 and the zoom lens 47. The zoom lens 47 is moved along the imaging optical axis (not shown) by the operation of the zoom operation unit 13b, thereby enlarging or reducing the observation target imaged by the imaging sensor 48.

The imaging sensor 48 is a color imaging sensor, and captures a reflected image of the observation target and outputs an image signal. As the imaging sensor 48, it is possible to use a charge coupled device (CCD) imaging sensor or a complementary metal oxide semiconductor (CMOS) imaging sensor. In the imaging sensor 48, color filters of three colors of a red (R) color filter, a green (G) color filter, and a blue (B) color filter shown in FIG. 10 are provided for each pixel. The imaging sensor 48 captures a reflected image of the observation target, and outputs an image signal of each color. That is, the imaging sensor 48 has an R pixel (red pixel) in which the R color filter is provided, a G pixel (green pixel) in that the G color filter is provided, and a B pixel (blue pixel) in that the B color filter is provided, and outputs an RGB image signal by outputting the image signal from each pixel.

As shown in Table 1, in the normal observation mode, the white light for normal observation is emitted to the observation target. Accordingly, the imaging sensor 48 receives light (reflected light, fluorescent light, or the like) corresponding to the violet light V and the blue light for normal observation B_{S} in the white light for normal observation using a B pixel, and outputs a blue image signal (hereinafter, referred to as a B image signal) corresponding to the blue wavelength band. Similarly, the imaging sensor 48 receives light corresponding to the green light G in the white light for normal observation using a G pixel and outputs a green image signal (hereinafter, referred to as a G image signal) corresponding to the green wavelength band, and receives light corresponding to the red light R in the white light for normal observation using an R pixel and outputs a red image signal (hereinafter, referred to as an R image signal) corresponding to the red wavelength band.

TABLE 1
Normal observation mode

Components of illumination light

| | V, B_{S} | G | R |
|---|---|---|---|
| Light receiving | B pixel | G pixel | R pixel |
| pixel | | | |
| Output image | B image | G image | R image |
| signal | signal | signal | signal |

As shown in Table 2, in the oxygen saturation observation mode, in a case in which the light source control unit 22 controls the light source unit 20 in the first light emission mode, the white light for normal observation is emitted to the observation target. Accordingly, the imaging sensor 48 receives light components corresponding to the violet light V and the blue light for normal observation B_{S} in the white light for normal observation using a B pixel, and outputs a first blue image signal (hereinafter, referred to as a B1 image signal). Similarly, the imaging sensor 48 receives light corresponding to the green light G in the white light for normal observation using a G pixel and outputs a first green image signal (hereinafter, referred to as a G1 image signal) corresponding to the green wavelength band, and receives light corresponding to the red light R in the white light for normal observation using an R pixel and outputs a first red image signal (hereinafter, referred to as an R1 image signal) corresponding to the red wavelength band.

TABLE 2
Oxygen saturation observation mode (first emission mode)

Components of illumination light

| | V, B_{S} | G | R |
|---|---|---|---|
| Light receiving | B pixel | G pixel | R pixel |
| pixel | | | |
| Output image | B1 image | G1 image | R1 image |
| signal | signal | signal | signal |

As shown in Table 3, in the oxygen saturation observation mode, in a case in which the light source control unit 22 controls the light source unit 20 in the second light emission mode, the white light for oxygen saturation measurement is emitted to the observation target. Accordingly, the imaging sensor 48 receives light corresponding to the measurement light B_{L} using a B pixel, and outputs a second blue image signal (hereinafter, referred to as a B2 image signal). Similarly, the imaging sensor 48 receives light corresponding to the green light G in the white light for oxygen saturation measurement using a G pixel and outputs a second green image signal (hereinafter, referred to as a G2 image signal) corresponding to the green wavelength band, and receives light corresponding to the red light R in the white light for oxygen saturation measurement using an R pixel and outputs a second red image signal (hereinafter, referred to as an R2 image signal) corresponding to the red wavelength band.

TABLE 3
Oxygen saturation observation mode (second emission mode)

Components of illumination light

| | B_{L} | G | R |
|---|---|---|---|
| Light receiving | B pixel | G pixel | R pixel |
| pixel | | | |
| Output image | B2 image | G2 image | R2 image |
| signal | signal | signal | signal |

Instead of the imaging sensor 48 that is a color imaging sensor of primary colors, a complementary color imaging sensor including complementary color filters of cyan (C), magenta (M), yellow (Y), and green (G) may be used. In the case of using the complementary color imaging sensor, image signals of four colors of CMYG are output. Therefore, by converting the image signals of four colors of CMYG into image signals of three colors of RGB by complementary color-primary color conversion, it is possible to obtain the same RGB image signals as in the imaging sensor 48. Instead of the imaging sensor 48, a monochrome sensor in which no color filter is provided may be used. In this case, the light source control unit 22 turns on the LEDs 20a to 20d in a time-division manner when necessary.

The image signal output from the imaging sensor 48 is transmitted to a CDS/AGC circuit 50. The CDS/AGC circuit 50 performs correlated double sampling (CDS) or automatic gain control (AGC) for the image signal that is an analog signal. The image signal transmitted through the CDS/AGC circuit 50 is converted into a digital image signal by an A/D converter 51. The digital image signal after A/D conversion is input to the processor device 16.

The processor device 16 includes an imaging control unit 52, an image signal acquisition unit 54, an image processing unit 61, and a display image signal generation unit 66. The image signal acquisition unit 54 includes a digital signal processor (DSP) 56, a noise reduction section 58, and a signal conversion section 59, and the image processing unit 61 includes a normal processing section 62 and a special processing section 63.

The imaging control unit 52 switches the observation mode by controlling the light source control unit 22 and the imaging sensor 48 in response to an input of a mode switching signal from the mode selector switch 13a. The imaging control unit 52 controls the type or amount of illumination light, the switching timing of illumination light, the length of exposure time of the imaging sensor 48, the value of a gain to be applied at the output of the image signal, imaging timing, and the like according to the light source control unit 22 and the imaging sensor 48.

Specifically, in a case in which the observation mode is a normal observation mode, the imaging control unit 52 controls the light source control unit 22 to always irradiate the observation target with the white light for normal observation, and controls the imaging sensor 48 to image the observation target for each fixed period (each imaging frame). Accordingly, in the observation mode, the image signal acquisition unit 54 acquires a B image signal, a G image signal, and an R image signal for each fixed period.

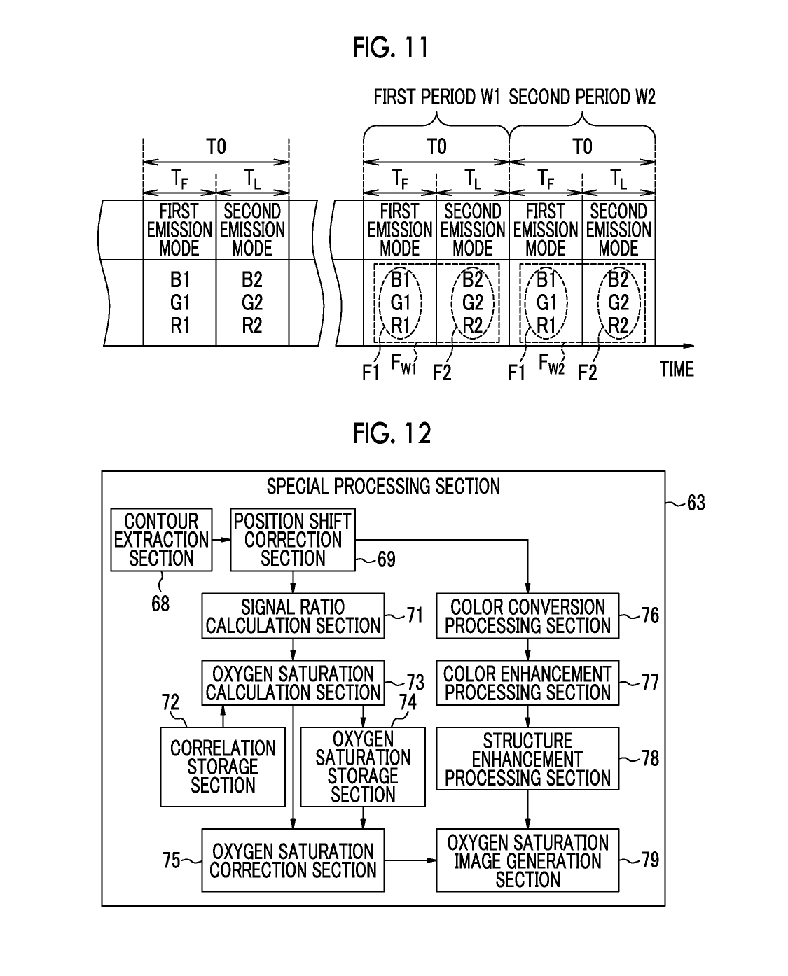

On the other hand, in a case in which the observation mode is an oxygen saturation observation mode, as shown in FIG. 11, the imaging control unit 52 controls the light source control unit 22 to alternate the first emission mode and the second emission mode for each fixed period (each imaging frame), and controls the imaging sensor 48 to image the observation target in each emission mode. Accordingly, the image signal acquisition unit 54 acquires a B1 image signal, a G1 image signal, an R1 image signal, a B2 image signal, a G2 image signal, and an R2 image signal.

In order to calculate the oxygen saturation, the B1 image signal, the G1 image signal, and the R1 image signal that are acquired in the first emission mode and the B2 image signal, the G2 image signal, and the R2 image signal that are acquired in the second emission mode are required. Accordingly, in the oxygen saturation observation mode, a “period T0” for which the B1 image signal, the G1 image signal, and the R1 image signal are acquired by imaging the observation target by emitting the white light for normal observation in the first emission mode and the B2 image signal, the G2 image signal, and the R2 image signal are acquired by imaging the observation target by emitting the white light for oxygen saturation measurement in the second emission mode is a unit period for acquiring an image signal required to generate one oxygen saturation image.

Hereinafter, a first half period T_{F} of the unit period T0 to acquire the B1 image signal, the G1 image signal, and the R1 image signal by imaging the observation target by emitting the white light for normal observation in the first emission mode is referred to as a “first timing T_{F}”. Similarly, a second half period T_{L} of the unit period T0 to acquire the B2 image signal, the G2 image signal, and the R2 image signal by imaging the observation target by emitting the white light for oxygen saturation measurement in the second emission mode is referred to as a “second timing T_{L}”. In addition, the B1 image signal, the G1 image signal, and the R1 image signal acquired at the first timing T_{F} are referred to as a “first group image signal F1”, and the B2 image signal, the G2 image signal, and the R2 image signal acquired at the second timing T_{L} are referred to as a “second group image signal F2”.

The latest unit period T0 to acquire the B1 image signal, the G1 image signal, the R1 image signal, the B2 image signal, the G2 image signal, and the R2 image signal is referred to as a “second period W2”, and the past unit period T0 to acquire the G1 image signal, the R1 image signal, the B2 image signal, the G2 image signal, and the R2 image signal immediately before the second period is referred to as a “first period W1”. In addition, the G1 image signal, the R1 image signal, the B2 image signal, the G2 image signal, and the R2 image signal acquired in the second period W2 are referred to as a “second period image signal F_{W2}”, and the G1 image signal, the R1 image signal, the B2 image signal, the G2 image signal, and the R2 image signal acquired in the first period W1 are referred to as a “first period image signal F_{W1}”.

The image signal acquisition unit 54 acquires an image signal as described above. In the normal observation mode, the image signal acquisition unit 54 acquires a B image signal, a G image signal, and an R image signal for each imaging frame. On the other hand, in the oxygen saturation observation mode, the image signal acquisition unit 54 acquires the first group image signal F1 (the B1 image signal, the G1 image signal, and the R1 image signal) and the second group image signal F2 (the B2 image signal, the G2 image signal, and the R2 image signal) alternately. Accordingly, the image signal acquisition unit 54 acquires the first period image signal F_{W1} by imaging the observation target, which is irradiated with illumination light, in the first period W1, and acquires the second period image signal F_{W2} by imaging the observation target in the second period W2 after the acquisition of the first period image signal F_{W1}.

The DSP 56 performs various kinds of signal processing, such as defect correction processing, offset processing, gain correction processing, linear matrix processing, gamma conversion processing, demosaic processing, and YC conversion processing, on the acquired image signals. In the defect correction processing, the signal of the defective pixel of the imaging sensor is corrected. In the offset processing, a dark current component is removed from the image signal after the defect correction processing, and the exact zero level is set. In the gain correction processing, the signal level of each image signal is adjusted by multiplying each of the RGB image signals after the offset processing by a specific gain. Linear matrix processing for increasing color reproducibility is performed on the image signal of each color after the gain correction processing. Then, the brightness or saturation of each image signal is adjusted by gamma conversion processing. Demosaic processing (also referred to as isotropic processing or synchronization processing) is performed on the image signal after the linear matrix processing, and the signal of missing color of each pixel is generated by interpolation. Through the demosaic processing, all pixels have signals of RGB colors. The DSP 56 performs YC conversion processing on each image signal after the demosaic processing, and outputs a brightness signal Y and color difference signals Cb and Cr to the noise reduction section 58.

The noise reduction section 58 performs noise reduction processing on the image signals after the demosaic processing or the like in the DSP 56 using, for example, a moving average method or a median filter method. The image signals after noise has been reduced are input to the signal conversion section 59, are reconverted into RGB image signals, and are input to the image processing unit 61.

The normal processing section 62 operates in the normal observation mode, and generates a normal image by performing color conversion processing, color enhancement processing, and structure enhancement processing on the RGB image signals. In the color conversion processing, color conversion processing, such as 3×3 matrix processing, gradation conversion processing, and three-dimensional look-up table (LUT) processing, is performed on the RGB image signals. The color enhancement processing is performed on the RGB image signals after the color conversion processing. The structure enhancement processing is a process for enhancing the structures of the observation target, such as superficial blood vessels or a pit pattern, and is performed on the RGB image signals after the color enhancement processing. As described above, a color image using the RGB image signals having been subjected to various kinds of image processing up to the structure enhancement processing is a normal image.

The special processing section 63 operates in the oxygen saturation observation mode, and calculates the oxygen saturation of the observation target using the image signals obtained in the oxygen saturation observation mode and generates an oxygen saturation image showing the oxygen saturation. As shown in FIG. 12, the special processing section 63 includes a contour extraction section 68, a position shift correction section 69, a signal ratio calculation section 71, a correlation storage section 72, an oxygen saturation calculation section 73, an oxygen saturation storage section 74, an oxygen saturation correction section 75, a color conversion processing section 76, a color enhancement processing section 77, a structure enhancement processing section 78, and an oxygen saturation image generation section 79.

The contour extraction section 68 extracts a contour component (information regarding an edge) of the observation target. More specifically, when the latest second period image signal F_{W2} is input, the contour extraction section 68 extracts a contour component at the first timing T_{S} using the first group image signal F1 included in the second period image signal F_{W2}. Similarly, the contour extraction section 68 extracts a contour component at the second timing T_{L} using the second group image signal F2 (the B2 image signal, the G2 image signal, and the R2 image signal) included in the second period image signal F_{W2}.

The position shift correction section 69 compares the contour component at the first timing T_{S} extracted by the contour extraction section 68 with the contour component at the second timing T_{L} extracted by the contour extraction section 68, and corrects the position shift of the first group image signal F1 or the second group image signal F2 to match the position of the observation target of the first group image signal F1 with the position of the observation target of the second group image signal F2. The position shift correction is for correcting the relative movement (mainly, parallel movement or rotation) of the observation target and the endoscope 12 that occurs between the first timing T_{F} to acquire the first group image signal F1 the second timing T_{L} to acquire the second group image signal F2. Therefore, artifacts due to the relative movement of the observation target and the endoscope 12 are substantially eliminated.

The signal ratio calculation section 71 calculates a signal ratio, which is to be used for the calculation of the oxygen saturation in the oxygen saturation calculation section 73, using the first group image signal F1 and the second group image signal F2 obtained after correcting the position shift in the position shift correction section 69. Specifically, the signal ratio calculation section 71 calculates a ratio between the B2 image signal included in the second group image signal F2 and the G1 image signal included in the first group image signal F1 (hereinafter, referred to as a signal ratio B2/G1) for each pixel. In addition, the signal ratio calculation section 71 calculates a ratio between the R1 image signal and the G1 image signal included in the first group image signal F1 (hereinafter, referred to as a signal ratio R1/G1) for each pixel.

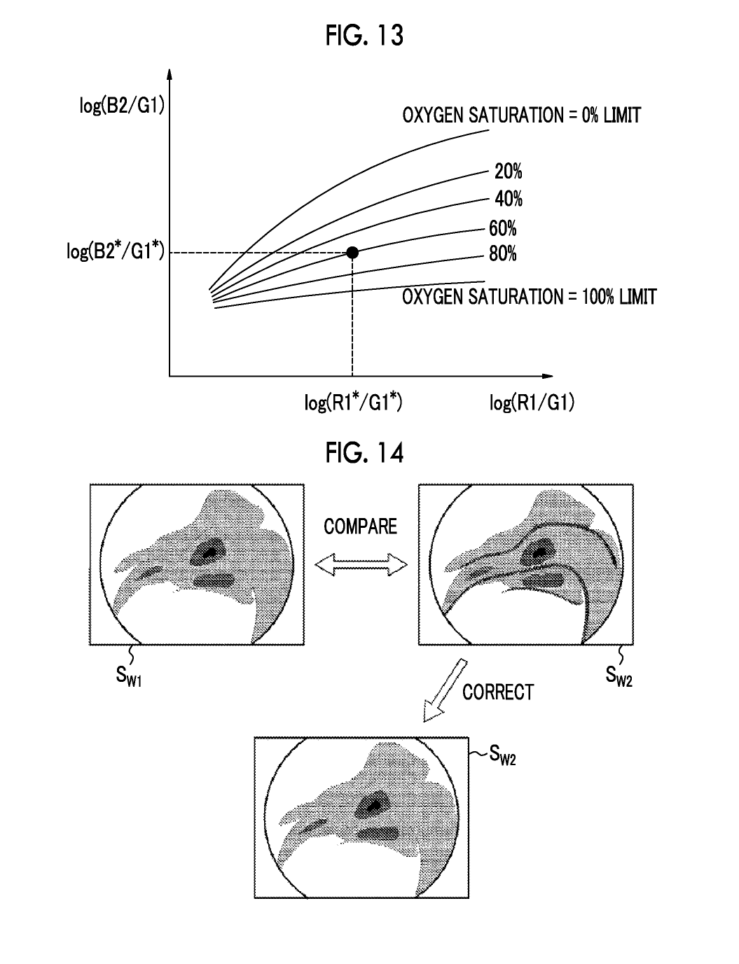

The correlation storage section 72 stores a correlation between each signal ratio calculated by the signal ratio calculation section 71 and the oxygen saturation. As shown in FIG. 13, this correlation is stored in a two-dimensional table that defines the isolines of oxygen saturation in a two-dimensional space. The position and shape of each isoline for the signal ratio are obtained in advance by physical simulation of light scattering. The distance between the isolines changes according to the signal ratio R1/G1 indicating the blood volume. In addition, the correlation between the signal ratio and the oxygen saturation is stored in a log scale.

This correlation is closely related to the absorption characteristics (refer to FIG. 8) or the light scattering characteristics of oxygenated hemoglobin or reduced hemoglobin. In the wavelength band of the measurement light B_{L} in which the difference between the absorption coefficient of oxygenated hemoglobin and the absorption coefficient of reduced hemoglobin is large, it is easy to handle the information of oxygen saturation. However, the B2 image signal corresponding to the measurement light B_{L} greatly depends on the blood volume as well as the oxygen saturation. Therefore, by using the signal ratio R1/G1, which is calculated from the G1 image signal that changes mainly depending on the blood volume and the R1 image signal having a low dependency on the oxygen saturation and the blood volume, in addition to the B2 image signal, it is possible to accurately calculate the oxygen saturation without there being dependency on the blood volume.

The oxygen saturation calculation section 73 calculates an oxygen saturation corresponding to the signal ratio B2/G1 and the signal ratio R1/G1 calculated by the signal ratio calculation section 71 with reference to the correlation stored in the correlation storage section 72. For example, in a case in which the signal ratio in a specific pixel is B2*/G1* and R1*/G1*, the oxygen saturation corresponding thereto is “60%” if the correlation is referred to (refer to FIG. 13). Accordingly, the oxygen saturation calculation section 73 calculates the oxygen saturation of the specific pixel as “60%”.

In addition, a case where the signal ratio B2/G1 and the signal ratio R1/G1 become extremely large or extremely small hardly occurs. That is, a case hardly occurs in which the combination of the signal ratio B2/G1 and the signal ratio R1/G1 exceeds the lower limit isoline indicating the oxygen saturation of 0% or becomes lower than the upper limit isoline indicating the oxygen saturation of 100%. Here, the oxygen saturation calculation section 73 calculates the oxygen saturation as 0% in a case in which the calculated oxygen saturation is lower than the lower limit isoline, and calculates the oxygen saturation as 100% in a case in which the calculated oxygen saturation exceeds the upper limit isoline.

In addition, the oxygen saturation calculation section 73 calculates the oxygen saturation in a sequential manner. Therefore, in the first period W1, the oxygen saturation calculation section 73 calculates the oxygen saturation S_{W1} in the first period W1 using the first period image signal F_{W1}. In addition, in the second period W2, the oxygen saturation calculation section 73 calculates the oxygen saturation S_{W2} in the second period W2 using the second period image signal F_{W2}. “Using the first period image signal F_{W1}” includes using the signal ratio B2/G1 and the signal ratio R1/G1 that are calculated using the first period image signal F_{W1}. Similarly, “using the second period image signal F_{W2}” includes using the signal ratio B2/G1 and the signal ratio R1/G1 that are calculated using the second period image signal F_{W2}.

The oxygen saturation storage section 74 stores the oxygen saturation calculated by the oxygen saturation calculation section 73. The oxygen saturation stored in the oxygen saturation storage section 74 is used by the oxygen saturation correction section 75. In the present embodiment, the oxygen saturation storage section 74 stores at least the oxygen saturation S_{W1} in the first period W1.

In a case in which the oxygen saturation S_{W2} in the latest second period W2 is calculated by the oxygen saturation calculation section 73, the oxygen saturation correction section 75 corrects the oxygen saturation S_{W2} in the latest second period W2 using the oxygen saturation S_{W1} in the past first period W1, as shown in FIG. 14. The correction of the oxygen saturation correction section 75 is performed for each pixel. More specifically, the oxygen saturation correction section 75 calculates the amount of change Δ in the oxygen saturation S_{W2} in the latest second period W2 with respect to the oxygen saturation S_{W1} in the past first period W1 for each pixel. Then, when the calculated amount of change Δ is less than a threshold value Th, the value of the oxygen saturation S_{W2} in the second period W2 is maintained. On the other hand, when the calculated amount of change Δ is equal to or greater than the threshold value Th, the value of the oxygen saturation S_{W2} in the second period W2 is corrected to the threshold value Th.

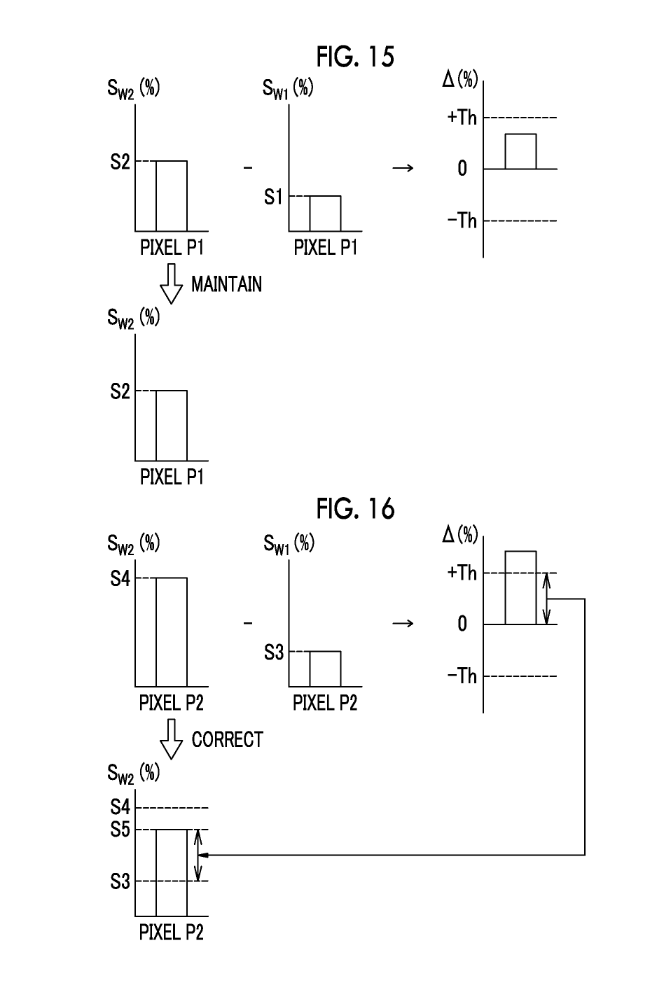

For example, as shown in FIG. 15, in a case in which the value of the oxygen saturation S_{W2} of a pixel P1 in the second period W2 is S2, the value of the oxygen saturation S_{W1} of the same pixel P1 in the first period W1 is S1, and the amount of change Δ (=S2−S1) is less than the threshold value Th (Δ<|Th|), the oxygen saturation correction section 75 maintains the value of the oxygen saturation S_{W2} of the pixel P1 in the second period W2 at the original value S2 even after correction. That is, in a case in which the amount of change Δ is small and the change in oxygen saturation can be regarded as a temporal or spatial change in original oxygen saturation corresponding to the characteristic change of the observation target, the oxygen saturation correction section 75 allows the change in oxygen saturation.

On the other hand, as shown in FIG. 16, in a case in which the value of the oxygen saturation S_{W2} of a pixel P2, which is different from the pixel P1, in the second period W2 is S4, the value of the oxygen saturation S_{W1} of the pixel P2 in the first period W1 is S3, and the amount of change Δ (=S4−S3) is equal to or greater than the threshold value Th (Δ<|Th|), the oxygen saturation correction section 75 corrects the value of the oxygen saturation S_{W2} of the pixel P2 in the second period W2 to a value S5 from the original value S4. The value S5 is a value obtained by adding the threshold value Th to the value S3 of the oxygen saturation S_{W1} in the first period W1 (S5=S3+|Th|). That is, in a case in which the amount of change is large and a possibility that the change in oxygen saturation will be artifacts is high, the oxygen saturation correction section 75 suppresses the amount of change Δ to the threshold value Th, and corrects the value of the oxygen saturation S_{W2} in the second period W2 to a value obtained as a result of setting the amount of change Δ to the threshold value Th. In this manner, the artifacts of the oxygen saturation S_{W2} in the second period W2 are reduced.

While calculating the oxygen saturation as described above, the special processing section 63 generates an image signal as a base (hereinafter, referred to as a base image signal) of the oxygen saturation image using the color conversion processing section 76, the color enhancement processing section 77, and the structure enhancement processing section 78. The color conversion processing section 76 performs color conversion processing, such as 3×3 matrix processing, gradation conversion processing, and three-dimensional LUT processing, on the first group image signal F1 (the B1 image signal, the G1 image signal, and the R1 image signal). The color enhancement processing section 77 performs color enhancement processing on the first group image signal F1 after the color conversion processing. The structure enhancement processing section 78 performs structure enhancement processing for enhancing the structures of the observation target, such as superficial blood vessels or a pit pattern, on the first group image signal F1 after the color enhancement processing. That is, the base image signal is formed by the B1 image signal, the G1 image signal, and the R1 image signal subjected to various kinds of image processing that are the same as in the normal processing section 62.

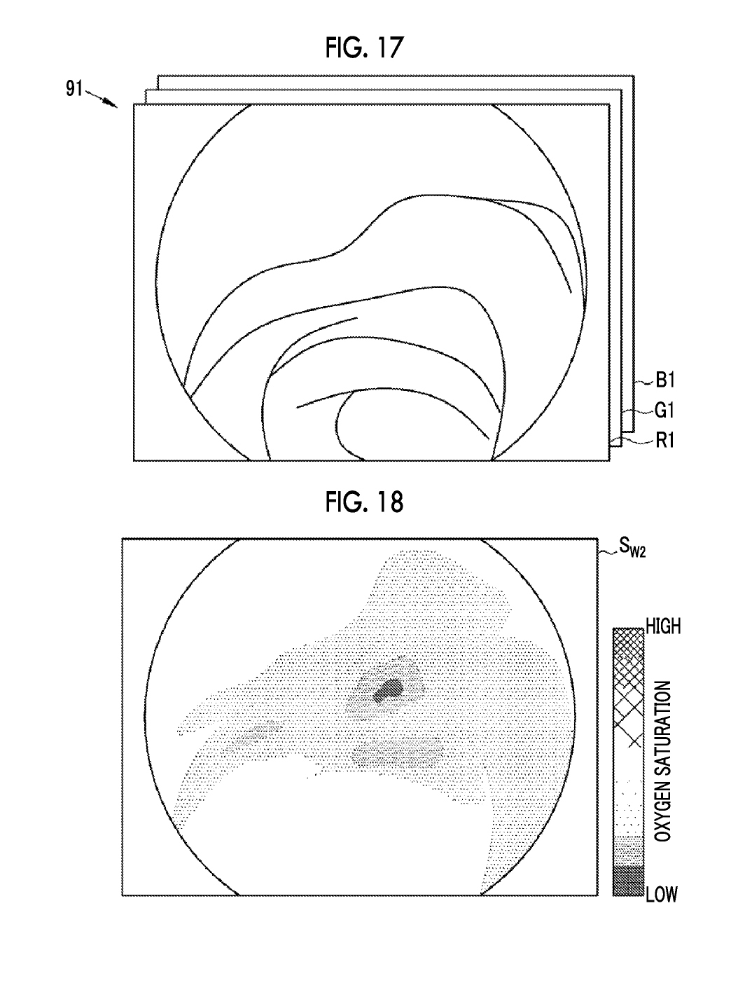

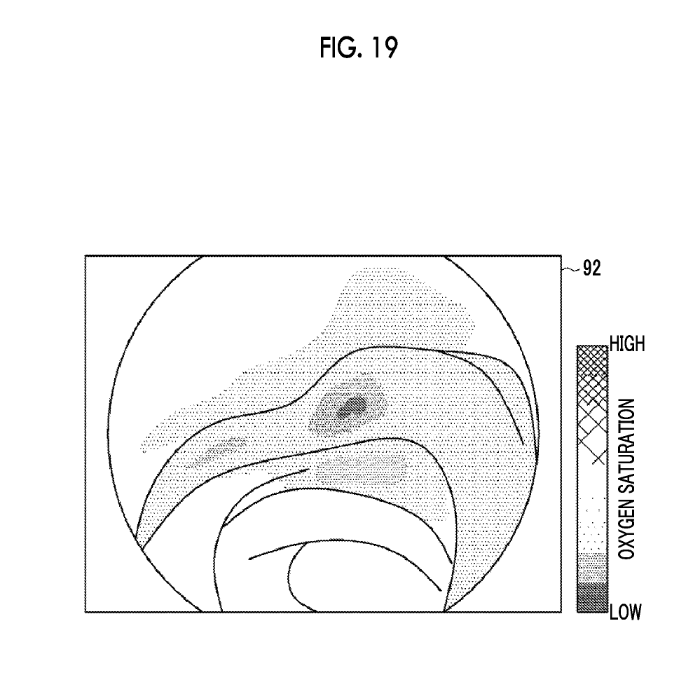

The oxygen saturation image generation section 79 generates an oxygen saturation image 92 showing the oxygen saturation of the observation target, as shown in FIG. 19, using a base image signal 91 shown in FIG. 17 and the oxygen saturation S_{W2} in the second period W2 obtained as a result of correcting the change equal to or greater than the threshold value Th to the threshold value Th by the oxygen saturation correction section 75 as shown in FIG. 18. More specifically, the oxygen saturation image 92 is generated by performing color conversion of the normal image generated from the base image signal 91, for each pixel, according to the value of the oxygen saturation S_{W2} in the second period W2 obtained as a result of correcting the change equal to or greater than the threshold value Th to the threshold value Th. Therefore, according to the oxygen saturation image 92, it is possible to observe the shape of the observation target or the like, and the color of the observation target of the oxygen saturation image 92 indicates the oxygen saturation.

As described above, the normal image generated by the normal processing section 62 in the normal observation mode and the oxygen saturation image 92 generated by the special processing section 63 in the oxygen saturation observation mode are input to the display image signal generation unit 66. The display image signal generation unit 66 converts the normal image or the oxygen saturation image 92 into a display format signal (display image signal), and inputs the display format signal to the monitor 18. As a result, the normal image or the oxygen saturation image 92 is displayed on the monitor 18.

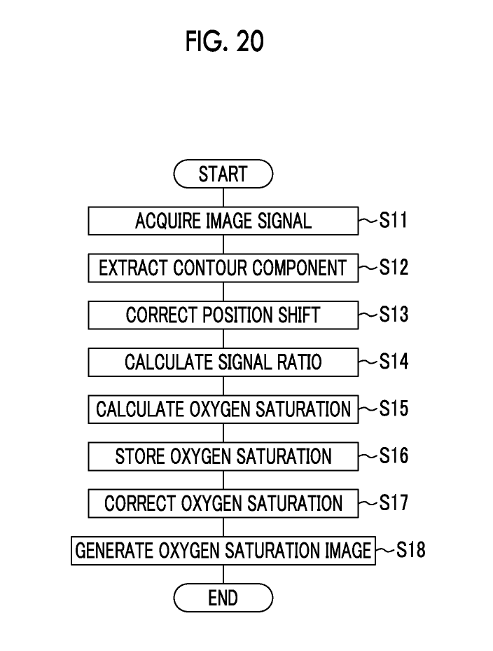

Next, a series of flow when the endoscope system 10 generates the oxygen saturation image 92 will be described along the flowchart shown in FIG. 20. First, when the observation mode is set to the oxygen saturation observation mode using the mode selector switch 13a, the imaging control unit 52 controls the light source control unit 22 to alternately emit the white light for normal observation in the first emission mode and the white light for oxygen saturation measurement in the second emission mode to the observation target. Then, according to the emission timing of such illumination light, the imaging control unit 52 controls the imaging sensor 48 to image the observation target, and the image signal acquisition unit 54 acquires the first group image signal F1 (the B1 image signal, the G1 image signal, and the R1 image signal) and the second group image signal F2 (the B2 image signal, the G2 image signal, and the R2 image signal) alternately and sequentially (S11).

While sequentially acquiring the first group image signal F1 and the second group image signal F2 as described above, when the image signal acquisition unit 54 acquires the first group image signal F1 and the second group image signal F2 that are acquired in the latest second period W2, the contour extraction section 68 in the special processing section 63 extracts a contour component of the first group image signal F1 and a contour component of the second group image signal F2 first (S12). Then, the position shift correction section 69 corrects the position shift of the observation target in the first group image signal F1 and the position shift of the observation target in the second group image signal F2 (S13). Then, the signal ratio calculation section 71 calculates the signal ratio B2/G1 and the signal ratio R1/G1 for calculating the oxygen saturation S_{W2} in the second period W2, for each pixel, using the first group image signal F1 and the second group image signal F2 in which a position shift has been corrected by the position shift correction section 69 (S14). Then, the oxygen saturation calculation section 73 calculates the oxygen saturation S_{W2} in the second period W2 corresponding to the signal ratio B2/G1 and the signal ratio R1/G1 calculated by the signal ratio calculation section 71 with reference to the correlation stored in the correlation storage section 72 (S15). The oxygen saturation S_{W2} calculated by the oxygen saturation calculation section 73 is sequentially stored in the oxygen saturation storage section 74 (S16).

As described above, in order to calculate the oxygen saturation S_{W2} in the second period W2, the first group image signal F1 acquired at the first timing T_{F} of the first half in the second period W2 and the second group image signal F2 acquired at the second timing T_{L} of the second half in the second period W2 are used in combination. For this reason, if there is a movement in the observation target between the first timing T_{F} and the second timing T_{L}, artifacts may be reflected on the calculated oxygen saturation. Among the movements of the observation target, the relative movement of the observation target and the endoscope 12 appearing as the parallel movement or rotation of the observation target is corrected by position shift correction. However, since there is a change (deformation) in the surface shape of the mucosa due to peristalsis or the like of the observation target, artifacts due to the change in the surface shape may be generated.

Therefore, the oxygen saturation correction section 75 corrects the oxygen saturation S_{W2} in the latest second period W2 using the oxygen saturation S_{W1} corresponding to the past first period W1 stored in the oxygen saturation storage section 74 (S17). By the correction of the oxygen saturation in the second period W2 in the oxygen saturation correction section 75, the amount of change Δ for the oxygen saturation S_{W1} in the first period W1 is suppressed to the threshold value Th or less. Accordingly, the original change in oxygen saturation corresponding to the characteristic change of the observation target is maintained, but artifacts corresponding to the movement (deformation or the like) of the observation target are suppressed to the threshold value Th at the maximum.

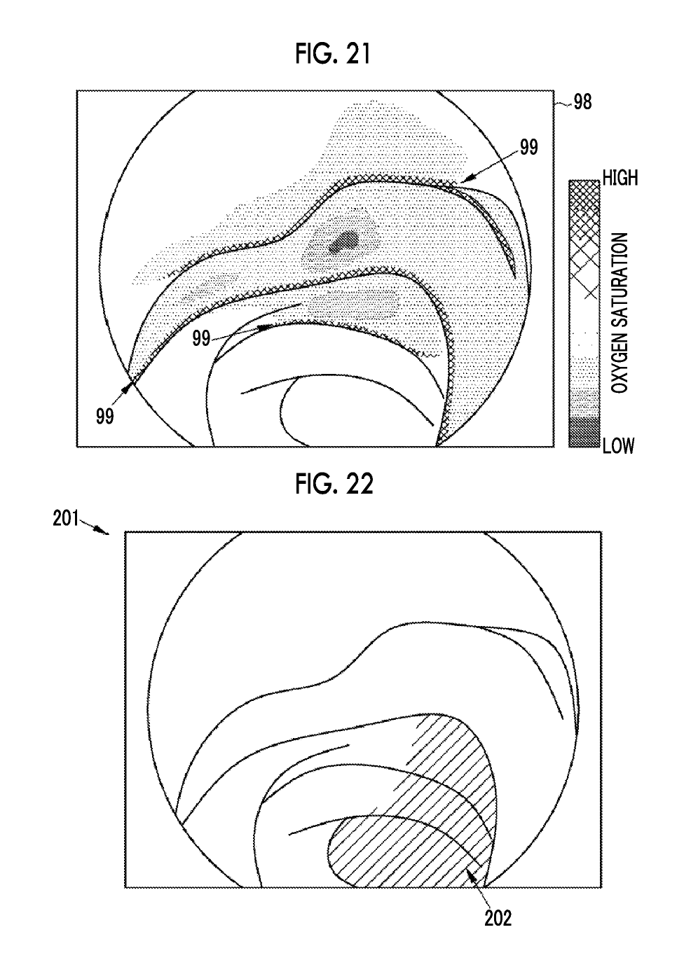

The oxygen saturation image generation section 79 generates the oxygen saturation image 92 using the oxygen saturation S_{W2} in the second period W2 obtained as a result of correcting the change equal to or greater than the threshold value Th to the threshold value Th by the oxygen saturation correction section 75 (S18). Accordingly, the endoscope system 10 can calculate an accurate oxygen saturation by reducing artifacts while leaving a temporal or spatial change in original oxygen saturation corresponding to the characteristic change of the observation target. For example, as shown in FIG. 21, in an oxygen saturation image 98 that is generated using the oxygen saturation S_{W2} in the latest second period W2 calculated by the oxygen saturation calculation section 73 without the correction of the oxygen saturation correction section 75, an artifact 99 is likely to be generated near the edge of the observation target. The oxygen saturation image 98 shown in FIG. 21 shows the artifact 99 of the high oxygen saturation appearing near the edge of the observation target. In contrast, in the oxygen saturation image 92 that is generated using the oxygen saturation S_{W2} in the latest second period W2 obtained as a result of correcting the change equal to or greater than the threshold value Th to the threshold value Th by the oxygen saturation correction section 75, such an artifact 99 is hardly generated (refer to FIG. 19).

In the first embodiment described above, the threshold value Th that is used for the comparison with the amount of change Δ in the oxygen saturation correction section 75 is fixed. However, the threshold value Th can be made variable. For example, it is preferable to set the threshold value Th based on the distribution of the oxygen saturation S_{W1} in the first period W1. In this case, it is preferable to change the threshold value Th according to the statistical value of the oxygen saturation S_{W1} in the first period W1, such as an average value, a median, a maximum value, or a minimum value. A statistical value for setting the threshold value Th may be calculated for the entire oxygen saturation S_{W1} in the first period W1, or the oxygen saturation S_{W1} in the first period W1 may be divided into a plurality of regions and the statistical value in each region may be calculated and used.

### Second Embodiment

An observation target is tubular, and illumination light from the endoscope 12 is difficult to reach an inner region of the observation target when viewed from the endoscope 12. In addition, since the observation target is uneven, a region where illumination light from the endoscope 12 is difficult to reach may be present depending on the shape of the observation target. For example, as a G1 image signal 201 shown in FIG. 22, in a rear region of the observation target distant from the endoscope 12 or a region that becomes a shadow of the unevenness of the observation target when viewed from the endoscope 12, the illumination intensity of illumination light is low. Accordingly, the rear region of the observation target distant from the endoscope 12 or the region that becomes a shadow of the unevenness of the observation target when viewed from the endoscope 12 may be a dark region (hereinafter, referred to as a dark region) 202 compared with the peripheral region. Thus, in a case in which the dark region 202 is generated in the G1 image signal 201, almost the same dark region is generated in the other first group image signal F1 (the B1 image signal and the R1 image signal) acquired at the first timing T_{L} together with the G1 image signal 201. In addition, in a case in which the dark region 202 is generated in the G1 image signal 201, almost the same dark region is generated in the second group image signal F2 (the B2 image signal, the G2 image signal, and the R2 image signal) acquired at the second timing T_{F} following the first timing T_{L} at which the G1 image signal 201 is acquired.

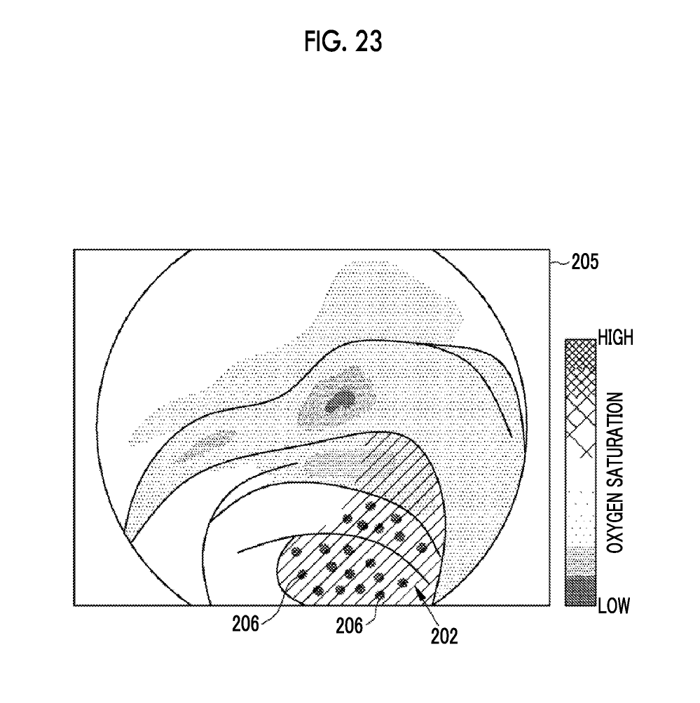

When the oxygen saturation S_{W2} in the second period W2 is calculated using the second period image signal F_{W2} in which the dark region 202 or the like has been generated as described above, even if artifacts due to the movement of the observation target are suppressed by correcting the oxygen saturation S_{W2} in the second period W2 in the oxygen saturation correction section 75, an artifact 206 that is not caused by the movement of the observation target may be generated in the dark region 202 as in an oxygen saturation image 205 shown in FIG. 23. The reason why the artifact 206 is generated is that the amount of random noise is relatively increased due to the insufficient amount of illumination light. In addition, when the artifact 206 is present which is not conspicuous in case of observing the oxygen saturation image 205 in a still image, if the oxygen saturation image 205 having the artifact 206 in the dark region 202 is observed in a moving image, the artifact 206 is moved or repeatedly blinks to become conspicuous. This may cause inconvenience at the time of observation.

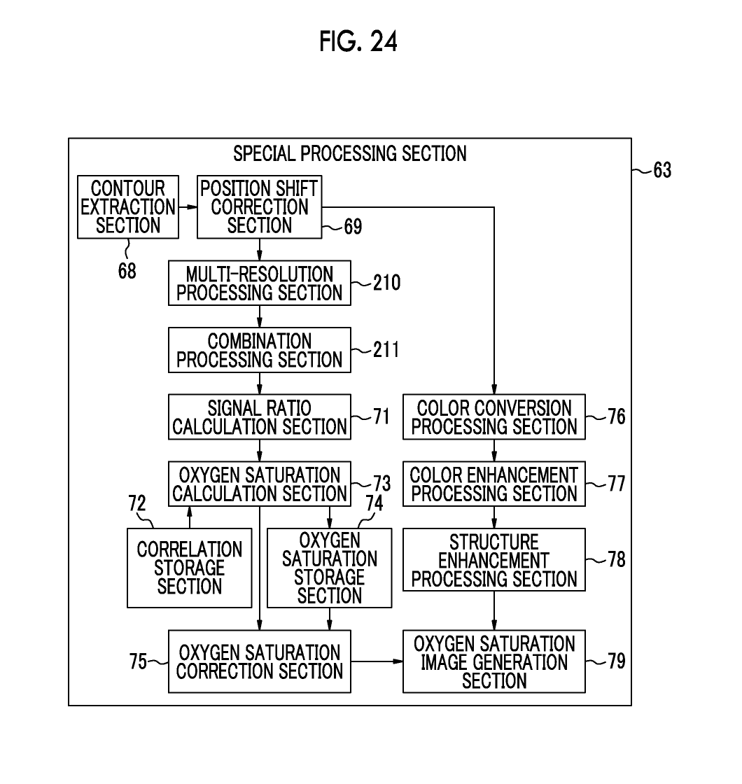

In order to reduce not only the artifact 99 due to the movement of the observation target but also the artifact 206 due to random noise, a multi-resolution processing section 210 and a combination processing section 211 are provided in the special processing section 63, as shown in FIG. 24.

The multi-resolution processing section 210 generates a plurality of image signals having different resolutions from the original image signal by performing multi-resolution processing on the image signal. More specifically, the multi-resolution processing section 210 generates an image signal having a lower resolution than the original image signal by performing low pass filter processing on the original image signal. In addition, the multi-resolution processing section 210 generates a plurality of image signals having lower resolutions than the original image signal by changing the cutoff frequency in the low pass filter processing. Accordingly, the multi-resolution processing section 210 generates a plurality of image signals (hereinafter, referred to as multi-resolution image signals) having different resolutions from one image signal. An image signal having an original resolution in which there is no change in the resolution before and after low pass filter processing is included in the multi-resolution image signals.

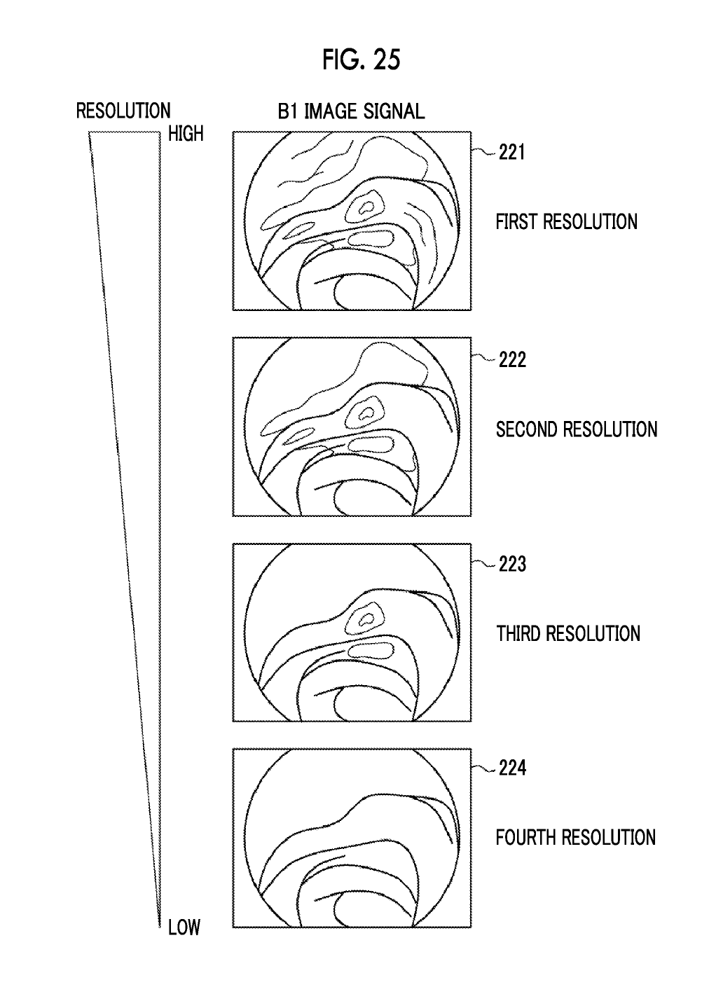

As in the first embodiment, the second period image signal F_{W2} obtained by correcting the position shift in the position shift correction section 69 is input to the multi-resolution processing section 210. Therefore, as shown in FIG. 25, first, the multi-resolution processing section 210 generates, for example, four kinds of multi-resolution image signals having first to fourth resolutions from the B1 image signal included in the first group image signal F1 acquired at the first timing T_{F} in the second period image signal F_{W2}. In the present embodiment, the first resolution is the same as resolution before low pass filter processing. Accordingly, the first resolution is the highest resolution, and the resolution decreases in the order of the second resolution, the third resolution, and the fourth resolution. Hereinafter, a multi-resolution image signal 221 having a first resolution that is generated from the B1 image signal by the multi-resolution processing section 210 is referred to as a “first resolution B1 image signal 221”, a multi-resolution image signal 222 having a second resolution that is generated from the B1 image signal by the multi-resolution processing section 210 is referred to as a “second resolution B1 image signal 222”, a multi-resolution image signal 223 having a third resolution that is generated from the B1 image signal by the multi-resolution processing section 210 is referred to as a “third resolution B1 image signal 223”, and a multi-resolution image signal 224 having a fourth resolution that is generated from the B1 image signal by the multi-resolution processing section 210 is referred to as a “fourth resolution B1 image signal 224”.

Similarly, the multi-resolution processing section 210 generates a first resolution G1 image signal, a second resolution G1 image signal, a third resolution G1 image signal, a fourth resolution G1 image signal, a first resolution R1 image signal, a second resolution R1 image signal, a third resolution R1 image signal, and a fourth resolution R1 image signal by performing multi-resolution processing on the G1 image signal and the R1 image signal that are included in the first group image signal F1 of the second period image signal F_{W2}.

Similarly, the multi-resolution processing section 210 generates multi-resolution image signals having first to fourth resolutions by performing multi-resolution processing on the second group image signal F2 (the B2 image signal, the G2 image signal, and the R2 image signal) of the second period image signal F_{W2}. The multi-resolution image signal having a first resolution that is generated from the B2 image signal is referred to as a first resolution B2 image signal in the same manner as described above.

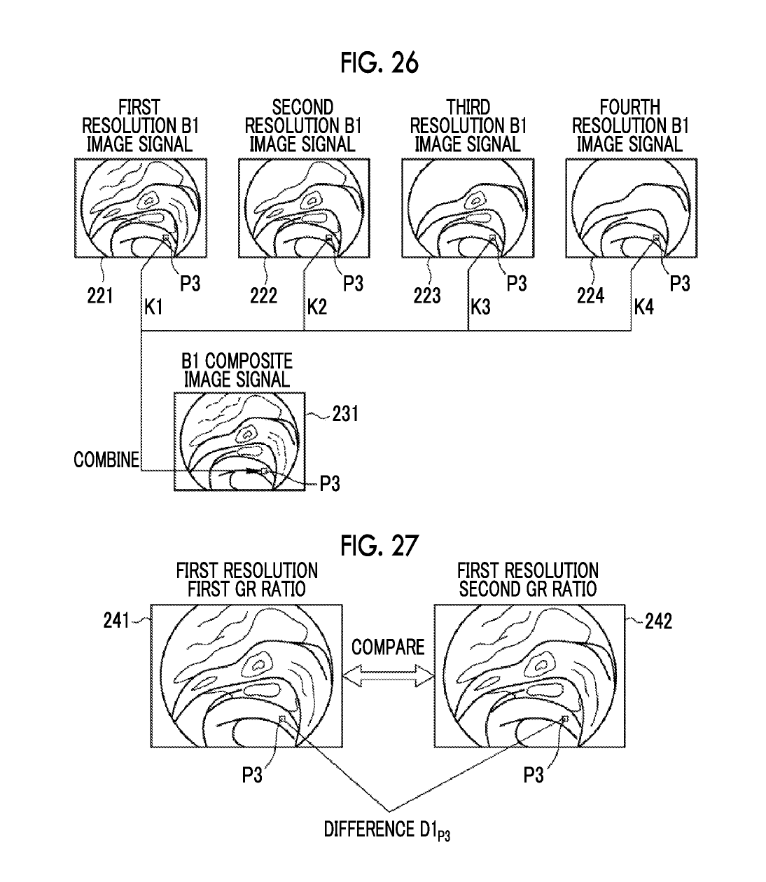

The combination processing section 211 compares the multi-resolution image signal generated from the first group image signal F1 with the multi-resolution image signal generated from the second group image signal F2 for each resolution and each pixel. Then, a “weighting” of each pixel and each resolution is set according to the comparison result, and the multi-resolution image signals are combined after the application of the set weighting, thereby generating a second period image composite image signal. For example, as shown in FIG. 26, in a case in which the weightings of specific pixels P3 of the first resolution B1 image signal 221, the second resolution B1 image signal 222, the third resolution B1 image signal 223, and the fourth resolution B1 image signal 224 are “K1”, “K2”, “K3”, and “K4”, respectively, the specific pixels P3 of the first resolution B1 image signal 221, the second resolution B1 image signal 222, the third resolution B1 image signal 223, and the fourth resolution B1 image signal 224 are combined after being multiplied by these weightings. By performing this for all pixels including the specific pixel P3, a B1 composite image signal 231 is generated. Similarly, a G1 composite image signal, an R1 composite image signal, a B2 composite image signal, a G2 composite image signal, and an R2 composite image signal are generated. The B1 composite image signal 231, the G1 composite image signal, the R1 composite image signal, the B2 composite image signal, the G2 composite image signal, and the R2 composite image signal are second period composite image signals.

A “weighting” is basically a different value for each pixel and each color of the image signal except for a case in which the “weighting” is the same value by chance. The combination processing section 211 calculates a “weighting” using the signal ratio between the multi-resolution image signal generated from the first group image signal F1 and the multi-resolution image signal generated from the second group image signal F2. Specifically, a “weighting” is set based on the closeness of the value of a color (ratio of G and R; hereinafter, referred to as a GR ratio) of the multi-resolution image signal generated from the first group image signal F1 and the value of a color (GR ratio) of the multi-resolution image signal generated from the second group image signal F2.

For example, in case of calculating the weighting of each resolution of the specific pixel P3, as shown in FIG. 27, the combination processing section 211 calculates a signal ratio (hereinafter, referred to as a first resolution first GR ratio) 241 between the first resolution G1 image signal and the first resolution R1 image signal and a signal ratio (hereinafter, referred to as a first resolution second GR ratio) 242 between the first resolution G2 image signal and the first resolution R2 image signal for each pixel, and compares these. Specifically, a difference D1_{P3} between the value of the specific pixel P3 having the first resolution first GR ratio and the value of the specific pixel P3 having the first resolution second GR ratio is calculated.

Similarly, a signal ratio (second resolution first GR ratio) between the second resolution G1 image signal and the second resolution R1 image signal and a signal ratio (second resolution second GR ratio) between the second resolution G2 image signal and the second resolution R2 image signal are calculated, and a difference D2_{P3} between the value of the specific pixel P3 having the second resolution first GR ratio and the value of the specific pixel P3 having the second resolution second GR ratio is calculated. Similarly for the third resolution and the fourth resolution, a difference D3_{P3} and a difference D4_{P3} are calculated.

The combination processing section 211 sets weightings “K1”, “K2”, “K3”, and “K4” that are inversely proportional to “D1_{P3}”, “D2_{P3}”, “D3_{P3}”, and “D4_{P3}”. That is, the combination processing section 211 increases the weighting as the closeness of resolutions between the GR ratio of the multi-resolution image signal generated from the first group image signal F1 acquired at the first timing T_{F} and the GR ratio of the multi-resolution image signal generated from the second group image signal F2 acquired at the second timing T_{L} increases. This is because the green light G and the red light R are the same at both the first timing T_{F} and the second timing T_{L}, and accordingly, the GR ratio of the multi-resolution image signal generated from the first group image signal F1 acquired at the first timing T_{F} and the GR ratio of the multi-resolution image signal generated from the second group image signal F2 acquired at the second timing T_{L} are close values if there is no random noise. Accordingly, increasing the weightings of resolutions, for which the GR ratios are close to each other, and combining the results as described above is equal to eliminating random noise. Accordingly, if the oxygen saturation image 92 is generated by calculating the oxygen saturation S_{W2} in the second period W2 as in the first embodiment using the second period composite image signal generated as described above by the combination processing section 211, no artifact 206 due to random noise appears in the oxygen saturation S_{W2} in the second period W2 and the oxygen saturation image 92.

In the second embodiment described above, the combination processing section 211 is provided, and the oxygen saturation is calculated using the second period composite image signal generated by the combination processing section 211. However, instead of using the second period composite image signal, the oxygen saturation S_{W2} in the second period W2 may be calculated using multi-resolution image signals in which the difference between the GR ratio of the multi-resolution image signal generated from the first group image signal F1 acquired at the first timing T_{F} and the GR ratio of the multi-resolution image signal generated from the second group image signal F2 acquired at the second timing T_{L} is the smallest and the resolution is the highest. For example, in a case in which the GR ratio of the specific pixel P3 is the smallest at the second resolution, the oxygen saturation S_{W2} of the specific pixel P3 in the second period W2 may be calculated using the second resolution B1 image signal, the second resolution G1 image signal, the second resolution R1 image signal, the second resolution B2 image signal, the second resolution G2 image signal, and the second resolution R2 image signal. Thus, even in a case in which the oxygen saturation S_{W2} in the second period W2 is calculated using the multi-resolution image signals in which the difference between the GR ratios is the smallest and the resolution is the highest, it is possible to reduce the artifact 206 due to random noise as in the second embodiment described above. In this case, since the combination processing section 211 does not perform combination processing, the combination processing section 211 functions as a GR ratio calculation section and a difference calculation section that calculates the difference between the GR ratios.

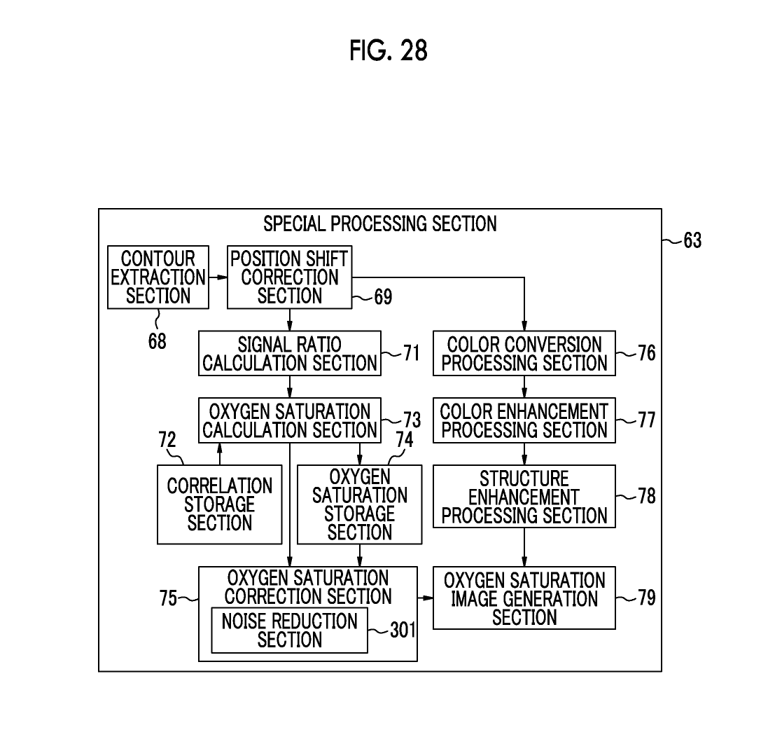

In addition, although the artifact 99 due to the movement of the observation target is reduced in the first embodiment and the artifact 206 due to the random noise of the dark region 202 is reduced in the second embodiment, it is also preferable to reduce artifacts due to other noise and the like. In this case, for example, as shown in FIG. 28, a noise reduction section 301 may be provided in the oxygen saturation correction section 75. The noise reduction section 301 performs so-called cyclic noise reduction processing (also referred to as three-dimensional noise reduction processing). The cyclic noise reduction processing is processing for reducing noise with low correlation between frames at random, for example, by averaging a plurality of image signals that are acquired in a short time and that are highly correlated with each other since the movement of the observation target is small. The noise reduction section 301 reduces the noise of the oxygen saturation S_{W2} in the latest second period W2 using the oxygen saturation S_{W1} in the first period W1 stored in the oxygen saturation storage section 74 or a plurality of oxygen saturations calculated in the past that include the oxygen saturation S_{W1} in the first period W1. By further applying the cyclic noise reduction processing of the noise reduction section 301, it is possible to reduce more artifacts than in the first and second embodiments described above.

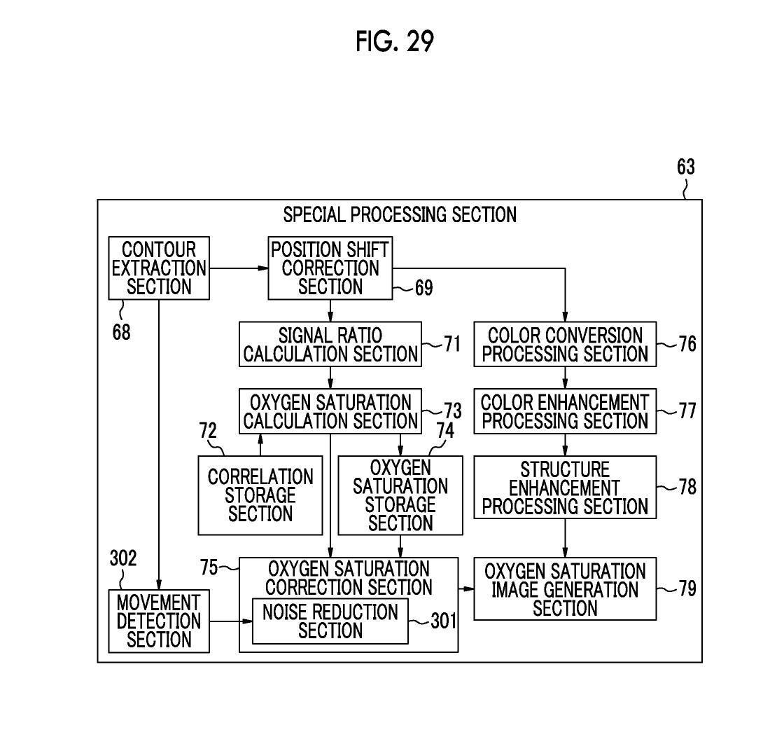

In addition, in a case in which the noise reduction section 301 is further used as described above, it is preferable to further provide a movement detection section 302 as shown in FIG. 29. The movement detection section 302 detects a relative movement between the observation target and the endoscope 12 using a contour component extracted by the contour extraction section 68, for example. More specifically, the movement detection section 302 detects the magnitude of the relative movement between the observation target and the endoscope 12. Then, the noise reduction section 301 changes the number of past oxygen saturations calculated before the second period W2, which are referred to for the noise reduction processing described above, according to the magnitude of the movement detected by the movement detection section 302. Specifically, the noise reduction section 301 increases the number of past oxygen saturations to be referred to for noise reduction processing as the magnitude of the relative movement between the observation target and the endoscope 12 increases. Typically, the cyclic noise reduction processing is effective in a case in which the movement of the observation target is small. Accordingly, in a case in which the movement of the observation target is large, the number of pieces of past data to be referred to should be reduced. However, in the case of the endoscope system 10 of the invention, the oxygen saturation correction section 75 reduces the artifact 99 due to the movement of the observation target. Therefore, it is possible to calculate the oxygen saturation especially with a small amount of noise by increasing the number of pieces of past oxygen saturation data to be referred to as the relative movement between the observation target and the endoscope 12 increases so that noise other than the artifact 99 due to the movement of the observation target can be reduced.

The movement detection section 302 in the modification example described above acquires a contour component from the contour extraction section 68, and detects the magnitude of the relative movement between the observation target and the endoscope 12. However, in a case in which a gyro sensor is provided in the distal portion 12d of the endoscope 12, the magnitude of the movement of the endoscope 12 may be detected based on the signal acquired from the gyro sensor.

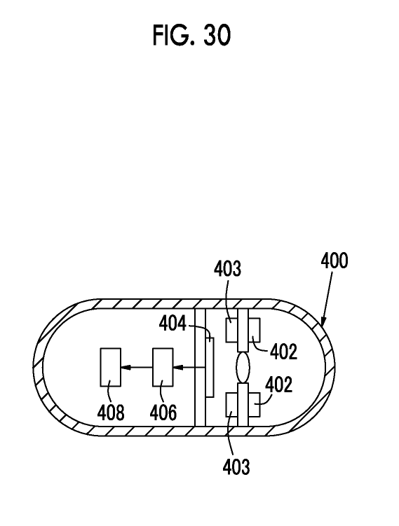

In addition, in the first and second embodiments and the modification example described above, the invention is implemented by using the endoscope system that performs observation by inserting the endoscope 12 including the imaging sensor 48 into the subject. However, the invention is also suitable for a capsule endoscope system. For example, as shown in FIG. 30, a capsule endoscope system includes at least a capsule endoscope 400 and a processor device (not shown).

The capsule endoscope 400 includes a light source 402, a control unit 403, an imaging sensor 404, an image processing unit 406, and a transmitting and receiving antenna 408. The light source 402 corresponds to the light source unit 20 and the band limiting unit 21. The control unit 403 functions similar to the light source control unit 22 and the imaging control unit 52. In addition, the control unit 403 can perform radio communication with the processor device of the capsule endoscope system through the transmitting and receiving antenna 408. Although the processor device of the capsule endoscope system is almost the same as the processor device 16 in the first and second embodiments and the modification example, the image processing unit 406 corresponding to the image signal acquisition unit 54 and the image processing unit 61 is provided in the capsule endoscope 400, and the generated oxygen saturation image 92 or the like is transmitted to the processor device through the transmitting and receiving antenna 408. The imaging sensor 404 is configured similar to the imaging sensor 48 in each of the embodiments described above.

What is claimed is:

1. An endoscope system, comprising:
an image signal acquisition unit that acquires a first period image signal by imaging an observation target, which is irradiated with illumination light, in a first period and that acquires a second period image signal by imaging the observation target in a second period after acquiring the first period image signal;
an oxygen saturation calculation unit that calculates an oxygen saturation of the observation target in the first period using the first period image signal and that calculates an oxygen saturation of the observation target in the second period using the second period image signal;
an oxygen saturation correction unit that calculates an amount of change in an oxygen saturation in the second period with respect to an oxygen saturation in the first period and that maintains a value of the oxygen saturation in the second period in a case in which the amount of change is less than a threshold value and corrects the oxygen saturation in the second period to a value obtained as a result of setting the amount of change to the threshold value in a case in which the amount of change is equal to or greater than the threshold value; and
an oxygen saturation image generation unit that generates an oxygen saturation image showing an oxygen saturation of the observation target using the oxygen saturation in the second period.

2. The endoscope system according to claim 1,
wherein the oxygen saturation correction unit sets the threshold value based on a distribution of the oxygen saturation in the first period.

3. The endoscope system according to claim 1, further comprising:
a multi-resolution processing unit that generates a plurality of multi-resolution image signals having different resolutions by performing multi-resolution processing on the second period image signal; and
a combination processing unit that generates a second period composite image signal by applying a weighting to each of the plurality of multi-resolution image signals,
wherein the oxygen saturation calculation unit calculates the oxygen saturation in the second period using the second period composite image signal.

4. The endoscope system according to claim 2, further comprising:
a multi-resolution processing unit that generates a plurality of multi-resolution image signals having different resolutions by performing multi-resolution processing on the second period image signal; and
a combination processing unit that generates a second period composite image signal by applying a weighting to each of the plurality of multi-resolution image signals,
wherein the oxygen saturation calculation unit calculates the oxygen saturation in the second period using the second period composite image signal.

5. The endoscope system according to claim 3,
wherein the second period image signal includes a first group image signal acquired at a first timing of the second period and a second group image signal acquired at a second timing, which is different from the first timing, of the second period, and
the combination processing unit compares a plurality of multi-resolution image signals generated from the first group image signal with a plurality of multi-resolution image signals generated from the second group image signal for each resolution, and performs combination processing by setting a larger weighting as the multi-resolution image signals having resolutions in which a difference between the multi-resolution image signal generated from the first group image signal and the multi-resolution image signal generated from the second group image signal is smaller.

6. The endoscope system according to claim 4,
wherein the second period image signal includes a first group image signal acquired at a first timing of the second period and a second group image signal acquired at a second timing, which is different from the first timing, of the second period, and
the combination processing unit compares a plurality of multi-resolution image signals generated from the first group image signal with a plurality of multi-resolution image signals generated from the second group image signal for each resolution, and performs combination processing by setting a larger weighting as the multi-resolution image signals having resolutions in which a difference between the multi-resolution image signal generated from the first group image signal and the multi-resolution image signal generated from the second group image signal is smaller.

7. The endoscope system according to claim 5,
wherein each of the first and second group image signals includes a green image signal corresponding to a green wavelength band and a red image signal corresponding to a red wavelength band, and
the combination processing unit compares ratios between the green image signal and the red image signal after multi-resolution processing for each resolution, and sets the weighting.

8. The endoscope system according to claim 6,
wherein each of the first and second group image signals includes a green image signal corresponding to a green wavelength band and a red image signal corresponding to a red wavelength band, and
the combination processing unit compares ratios between the green image signal and the red image signal after multi-resolution processing for each resolution, and sets the weighting.

9. The endoscope system according to claim 1, further comprising:
a multi-resolution processing unit that generates a plurality of multi-resolution image signals having different resolutions by performing multi-resolution processing on the second period image signal,
wherein the oxygen saturation calculation unit calculates the oxygen saturation in the second period using the multi-resolution image signal having a specific resolution of the plurality of multi-resolution image signals.

10. The endoscope system according to claim 2, further comprising:
a multi-resolution processing unit that generates a plurality of multi-resolution image signals having different resolutions by performing multi-resolution processing on the second period image signal,
wherein the oxygen saturation calculation unit calculates the oxygen saturation in the second period using the multi-resolution image signal having a specific resolution of the plurality of multi-resolution image signals.

11. The endoscope system according to claim 9,
wherein the second period image signal includes a first group image signal acquired at a first timing of the second period and a second group image signal acquired at a second timing, which is different from the first timing, of the second period, and
the oxygen saturation calculation unit calculates the oxygen saturation in the second period using the multi-resolution image signals in which a difference between the multi-resolution image signal generated from the first group image signal and the multi-resolution image signal generated from the second group image signal is the smallest and resolutions are the highest.

12. The endoscope system according to claim 10,
wherein the second period image signal includes a first group image signal acquired at a first timing of the second period and a second group image signal acquired at a second timing, which is different from the first timing, of the second period, and
the oxygen saturation calculation unit calculates the oxygen saturation in the second period using the multi-resolution image signals in which a difference between the multi-resolution image signal generated from the first group image signal and the multi-resolution image signal generated from the second group image signal is the smallest and resolutions are the highest.

13. The endoscope system according to claim 1, further comprising:
an oxygen saturation storage unit that stores a plurality of oxygen saturations including the oxygen saturation in the first period and calculated before the second period; and
a noise reduction unit that reduces noise of the oxygen saturation in the second period using a plurality of oxygen saturations stored in the oxygen saturation storage unit.

14. The endoscope system according to claim 2, further comprising:
an oxygen saturation storage unit that stores a plurality of oxygen saturations including the oxygen saturation in the first period and calculated before the second period; and
a noise reduction unit that reduces noise of the oxygen saturation in the second period using a plurality of oxygen saturations stored in the oxygen saturation storage unit.

15. The endoscope system according to claim 3, further comprising:
an oxygen saturation storage unit that stores a plurality of oxygen saturations including the oxygen saturation in the first period and calculated before the second period; and
a noise reduction unit that reduces noise of the oxygen saturation in the second period using a plurality of oxygen saturations stored in the oxygen saturation storage unit.

16. The endoscope system according to claim 4, further comprising:
an oxygen saturation storage unit that stores a plurality of oxygen saturations including the oxygen saturation in the first period and calculated before the second period; and
a noise reduction unit that reduces noise of the oxygen saturation in the second period using a plurality of oxygen saturations stored in the oxygen saturation storage unit.

17. The endoscope system according to claim 13, further comprising:
a movement detection unit that detects a relative movement between the observation target and an endoscope,
wherein the noise reduction unit increases the number of oxygen saturations used for reduction of the noise as the movement detected by the movement detection unit increases.

18. The endoscope system according to claim 1, further comprising:
a position shift correction unit that corrects a position shift between the oxygen saturation in the second period and the oxygen saturation in the first period.

19. A processor device, comprising:
an image signal acquisition unit that acquires a first period image signal by imaging an observation target, which is irradiated with illumination light, in a first period and that acquires a second period image signal by imaging the observation target in a second period after acquiring the first period image signal;
an oxygen saturation calculation unit that calculates an oxygen saturation of the observation target in the first period using the first period image signal and that calculates an oxygen saturation of the observation target in the second period using the second period image signal;
an oxygen saturation correction unit that calculates an amount of change in an oxygen saturation in the second period with respect to an oxygen saturation in the first period and that maintains a value of the oxygen saturation in the second period in a case in which the amount of change is less than a threshold value and corrects the oxygen saturation in the second period to a value obtained as a result of setting the amount of change to the threshold value in a case in which the amount of change is equal to or greater than the threshold value; and
an oxygen saturation image generation unit that generates an oxygen saturation image showing an oxygen saturation of the observation target using the oxygen saturation in the second period.

20. An operation method of the endoscope system using the endoscope system according to claim 1, comprising:
a step of causing an image signal acquisition unit to acquire a first period image signal by imaging an observation target, which is irradiated with illumination light, in a first period and to acquire a second period image signal by imaging the observation target in a second period after acquiring the first period image signal;
a step of causing an oxygen saturation calculation unit to calculate an oxygen saturation of the observation target in the first period using the first period image signal and to calculate an oxygen saturation of the observation target in the second period using the second period image signal;
a step of causing an oxygen saturation correction unit to calculate an amount of change in an oxygen saturation in the second period with respect to an oxygen saturation in the first period and to maintain a value of the oxygen saturation in the second period in a case in which the amount of change is less than a threshold value and correct the oxygen saturation in the second period to a value obtained as a result of setting the amount of change to the threshold value in a case in which the amount of change is equal to or greater than the threshold value; and
a step of causing an oxygen saturation image generation unit to generate an oxygen saturation image showing an oxygen saturation of the observation target using the oxygen saturation in the second period.
